(19) United States
(12) Patent Application Publication
(10) Pub. No.: US 2026/0341408 A1
(43) Pub. Date: Oct. 29, 2026

(54) CELL-FREE DNA SEQUENCE DATA ANALYSIS TECHNIQUES FOR ESTIMATING FETAL FRACTION AND PREDICTING PREECLAMPSIA

(71) Applicants: FRED HUTCHINSON CANCER CENTER, Seattle, WA (US); UNIVERSITY OF WASHINGTON, Seattle, WA (US)

(72) Inventors: Mohamed ADIL, Seattle, WA (US); Gavin HA, Seattle, WA (US); Jonathan Brett REICHEL, Seattle, WA (US); Christina M. LOCKWOOD, Seattle, WA (US); Raj SHREE, Seattle, WA (US)

(73) Assignees: FRED HUTCHINSON CANCER CENTER, Seattle, WA (US); UNIVERSITY OF WASHINGTON, Seattle, WA (US)

(21) Appl. No.: 19/106,304

(22) PCT Filed: Aug. 25, 2023
(86) PCT No.: PCT/US2023/072909

§ 371 Date: Feb. 25, 2025

Related U.S. Application Data
(60) Provisional application No. 63/401,547, filed on Aug. 26, 2022.

(51) Int. Cl. G16B 30/00; G16B 40/20; G16H 10/40; G16H 50/20

(52) U.S. Cl. CPC G16B 30/00; G16B 40/20; G16H 10/40; G16H 50/20

(57) ABSTRACT

In some embodiments, a computer-implemented method of enhancing sequence read data from a cell-free DNA (cfDNA) sample from a subject for predicting a pregnancy-related condition is provided. A computing system determines a coverage profile based on sequence read data for a plurality of informative sites associated with specific tissue types, cell types, or cell states. The computing system generates a prediction of a presence of or an absence of the pregnancy-related condition by providing at least features from a set of features based on a predicted fetal fraction and a set of features based on the coverage profile as input to at least one machine learning model trained to predict a probability of future onset of the pregnancy-related condition based on the features. In some embodiments, a computer-implemented method of enhancing sequence read data for predicting fetal fraction is provided.

### CROSS-REFERENCE TO RELATED APPLICATION

This application claims the benefit of Provisional Application No. 63/401,547, filed Aug. 26, 2022, the entire disclosure of which is hereby incorporated by reference herein for all purposes.

### BACKGROUND

Liquid biopsy continues to mature in importance for molecular diagnostics and precision medicine. Plasma-derived cell-free DNA (cfDNA) is a robust analyte for clinical application with noninvasive prenatal screening (NIPS) for aneuploidy detection being the most clinically utilized application. NIPS is a clinical test by which individual pregnancies are screened for the risk of carrying a fetus with a genetic abnormality (such as Down Syndrome, also known as Trisomy 21). NIPS most commonly refers to genetic screening performed from sequencing of cfDNA, however, it can more broadly refer to any screening methodology that uses a maternal blood sample. NIPS outperforms conventional biochemical prenatal screening, and is now recommended as a first line aneuploidy screening tool for both high- and low-risk pregnancies. Aneuploidy is a genetic disorder where the total number of chromosomes does not equal 46.

One quality control step often present in any NIPS platform is determination of the fraction of cfDNA arising from the placenta among the total amount of cfDNA, often termed the fetal fraction (FF). Samples with low FF (typically <4%) may yield false negative results but are also associated with increased rates of aneuploidy. While the FF for samples from pregnancies with male fetuses can be precisely calculated by quantifying the chromosome-Y fetal fraction (ChrY-FF), the FF for non-male fetuses lack similar accuracy from shallow whole genome sequencing (WGS) data, and tend to rely on heuristic-based estimations.

Preeclampsia (PEP) is a pregnancy-specific condition characterized by high blood pressure (hypertension) and evidence of other organ system dysfunction (such as renal, liver, hematologic, or neurologic) that is diagnosed at or after 20 weeks gestation. PEP, which is associated with substantial morbidity and mortality for the maternal-fetal dyad, is rooted in placental dysfunction. However, early pregnancy prediction has remained elusive. PEP prediction algorithms have not been widely adopted, particularly in the United States, due to suboptimal positive predictive values, with current reliance solely on clinical risk factors. An analysis of 2019 birth certificate data (>3.6 million births) demonstrated that approximately 50.4% of pregnancies would screen positive for being at high-risk for developing a hypertensive disorder of pregnancy (HDP) based on 2021 United States Preventive Services Task Force (USPSTF) recommendations (≥2 moderate risk factors or ≥1 high-risk factor) meeting criteria for low-dose aspirin prophylaxis. Importantly, only 10.5% of those flagged as “high-risk” developed HDP. Conversely, of the 49.5% of pregnancies that did not meet USPSTF criteria as candidates for low dose aspirin prophylaxis, 5.7% (over 100,000 pregnancies) developed HDP. Advancement beyond reliance on crude clinical risk stratification is needed to more precisely identify those pregnancies at highest risk for HDP/PEP. Additionally, HDP presents on a spectrum, with the disproportionate maternal and fetal burden being faced by those that develop HDP at early gestational ages as this tends to be more severe for the mother and often leads to a premature birth for the offspring. It is possible that interventions beyond low dose aspirin, such as strict blood pressure control, may limit the burden of PEP, for which precise early screening tools are needed.

BRIEF SUMMARY

In some embodiments, a computer-implemented method of enhancing sequence read data from a cell-free DNA (cfDNA) sample from a subject for predicting a pregnancy-related condition is provided. A computing system receives sequence read data. The sequence read data includes a plurality of fragment reads. The computing system determines a coverage profile based on the sequence read data for a plurality of informative sites associated with specific tissue types, cell types, or cell states. The computing system determines a predicted fetal fraction based on the sequence read data. The computing system determines a set of features based on the predicted fetal fraction and a set of features based on the coverage profile. The computing system generates a prediction of a presence of or an absence of the pregnancy-related condition by providing at least features from the set of features based on the predicted fetal fraction and the set of features based on the coverage profile as input to at least one machine learning model trained to predict a probability of future onset of the pregnancy-related condition based on the features.

In some embodiments, a computer-implemented method of enhancing sequence read data from a cell-free DNA (cfDNA) sample from a subject for predicting a fetal fraction of cfDNA contained in the sample is provided. A computing system receives sequence read data. The sequence read data includes a plurality of fragment reads. The computing system determines a coverage profile based on the sequence read data for a plurality of informative sites associated with fetal fraction determination. The computing system determines a set of features based on the coverage profile. The computing system generates a prediction of a fetal fraction by providing features from the set of features as input to at least one machine learning model trained to predict the fetal fraction based on the features.

In some embodiments, a computing system configured to perform a method as described above is provided.

In some embodiments, a non-transitory computer-readable medium is provided. The computer-readable medium has instructions stored thereon that, in response to execution by a computing system, cause the computing system to perform a method as described above.

### BRIEF DESCRIPTION OF THE SEVERAL VIEWS OF THE DRAWINGS

Non-limiting and non-exhaustive embodiments of the disclosure are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described. To easily identify the discussion of any particular element or act, the most significant digit or digits in a reference number refer to the figure number in which that element is first introduced.

FIG. 1 is a block diagram that illustrates aspects of a non-limiting example embodiment of a computing system configured to estimate FF and/or detect pregnancy-related conditions based on sequence read data, according to various aspects of the present disclosure.

FIG. 2 is a flowchart that illustrates a non-limiting example embodiment of a method of enhancing sequence read data from a cfDNA sample from a subject for predicting a pregnancy-related condition, according to various aspects of the present disclosure

FIG. 3 is a flowchart that illustrates a non-limiting example embodiment of a method for enhancing sequence read data from a cfDNA sample from a subject for predicting a FF of cfDNA contained in the sample, according to various aspects of the present disclosure.

FIG. 4 is a flowchart that illustrates a non-limiting example embodiment of a procedure for determining a coverage profile for a plurality of informative sites in sequence read data according to various aspects of the present disclosure.

FIG. 5 is a schematic illustration of a non-limiting example embodiment of a framework for analyzing NIPS data, according to various aspects of the present disclosure.

FIG. 6A and FIG. 6B are charts showing nucleosome profiles characterized at accessible sites in a training cohort of 1418 cfDNA samples, and illustrate that immune cells and placental cells had the strongest signals of accessibility at the nucleosome depleted region (NDR) as indicated by decrease in fragment coverage.

FIG. 7, FIG. 8, and FIG. 9 illustrate performance achieved for a regularized linear regression model that was built to estimate the FF from NIPS data on a training cohort, a validation cohort, and an external cohort, respectively.

FIG. 10A and FIG. 10B are schematic illustrations summarizing a study testing nucleosome profiling of cfDNA from NIPS to predict preeclampsia, according to various aspects of the present disclosure.

FIG. 10C and FIG. 10D are charts illustrating performance of using nucleosome profiling to predict FF in the study of FIG. 10A-FIG. 10B.

FIG. 10E-FIG. 10I are charts that illustrate further aspects of the data used and results obtained in the study of FIG. 10A-FIG. 10B.

FIG. 11 is a chart that illustrates results of coverage profiling, showing a trend of accessibility of sites specific to erythroid and endothelial cells decreasing by trimesters, collectively supporting that the majority of cfDNA originates from hematopoietic cell types.

FIG. 12 is a chart that illustrates that accessibility at sites associated with immune cells were strongest for non-pregnant women and decreased by trimesters in NIPS samples.

FIG. 13 is a chart that illustrates that the accessibility at placenta tissue sites in NIPS samples increased by trimester while in 20 non-pregnant donor samples had significantly lower accessibility.

FIG. 14A and FIG. 14B are charts that illustrate an inverse trend observed for sub-nucleosome (35-80 bp) fragments.

FIG. 15A-FIG. 15F include illustrations of analysis of CfDNA for an additional 16 sets of tissues derived from DNase I hypersensitivity sites sequencing (DNase-seq), and show similarly strong accessibility trends in immune and placenta tissue types.

FIG. 16A and FIG. 16B are charts that illustrate cfDNA nucleosome accessibility at open chromatin sites across tissue types, for the assessment of whether the signals are associated with maternal physiology according to various aspects of the present disclosure.

FIG. 17A-FIG. 17J are charts that illustrate correlations between various tissue-specific signals and gestational age in weeks as determined from the study illustrated in FIG. 16A and FIG. 16B.

FIG. 18A-FIG. 18J are charts that illustrate correlations between various tissue-specific signals and BMI as determined from the study illustrated in FIG. 16A and FIG. 16B.

FIG. 19A, FIG. 19B, FIG. 20A, and FIG. 20B illustrate investigations of whether the nucleosome profiles of tissue-specific sites can inform the FF, according to various aspects of the present disclosure.

FIG. 21A-FIG. 21J and FIG. 22A-FIG. 22J are charts that illustrate results of the investigations of FIG. 19A-FIG. 19B and FIG. 20A-FIG. 20B into whether coverage for various tissue-specific sites is correlated with ChrY-FF.

FIG. 23A and FIG. 23B are charts that illustrate correlations between TFBS accessibility for some TFs and ChrY-FF.

FIG. 24A and FIG. 24B are charts that illustrate coverage related to a GRHL2 TF and a LYL1 TF, respectively.

FIG. 24B illustrates an aspect of the subject matter in accordance with one embodiment.

FIG. 25A and FIG. 25B are charts that illustrate effects of sequencing depth on Griffin-FF according to various aspects of the present disclosure.

### DETAILED DESCRIPTION

Differentiation of placental-derived cfDNA in a background of maternal-derived cfDNA has presented challenges. However, advances in our understanding of epigenetics, DNA fragmentation patterns, and cfDNA topology have uncovered unique tissue-specific signatures that can be leveraged and have been used in the related fields of cancer and transplant biology. Nucleosome positioning varies by cell type and the resultant fragmentation pattern of cfDNA contains evidence of the epigenetic and transcriptional landscape of the original cell. Given that the FF represents DNA arising from the placenta, this biologic material coupled with advanced computational tools assessing nucleosome profiling can inform not only aneuploidy status, but placental health and function more broadly.

Nucleosome profiling infers the position of nucleosomes directly from analysis of coverage profiles in cfDNA sequencing data. More generally, nucleosome profiling includes consideration of mono-nucleosomes, dinucleosomes, and sub-nucleosomes. A coverage profile indicates sequencing coverage values at the resolution of each base pair within a region, window, or bin of the genome. The region is centered at an informative site. The size of the region can vary in range. The coverage profile can also be determined by aggregating multiple regions, windows, or bins across the genome. The coverage is typically processed and/or normalized and/or corrected for sequencing biases.

Given the presumed placental etiology of most severe and early-onset cases of PEP, non-invasive methods of placental interrogation can advance our understanding of early pregnancy placental alterations that contribute to the later pregnancy phenotype of PEP. Since placental-derived cfDNA contains within it the genetic and epigenetic signature of the placenta, it is a promising target for non-invasive placental sampling that can be leveraged for PEP screening.

The present disclosure utilizes novel techniques for nucleosome profiling of cfDNA via analysis of coverage profiles in maternal plasma for a) sex-independent determination of FF; and b) prediction of PEP. Using ChrY-FF determined from maternal cfDNA samples harboring male fetuses as ground truth, we trained a supervised regression model that uses nucleosome profiling to predict FF in a manner that is not impacted by the sex of the fetus.

Previous studies have shown that deep whole genome sequencing (WGS) of cfDNA reveals the footprints of in-vivo nucleosome positions along the genome. This yields an epigenetic molecular profile capable of identifying and differentiating between the originating tissue types. It is observed that cfDNA fragment size distribution in plasma has a median length of 167 bp, consistent with nucleosome protection during the process of DNA fragmentation from cell turnover. WGS of cfDNA can reveal these nucleosomal footprints along the genome, which are cell- and tissue-specific. The present disclosure provides novel techniques for nucleosome profiling of WGS data generated from maternal plasma cfDNA to quantify the FF in a sex-independent manner. The present disclosure also provides novel machine-learning techniques that use a model for the prediction of early-onset PEP and PEP complicated by preterm birth from an early pregnancy sample.

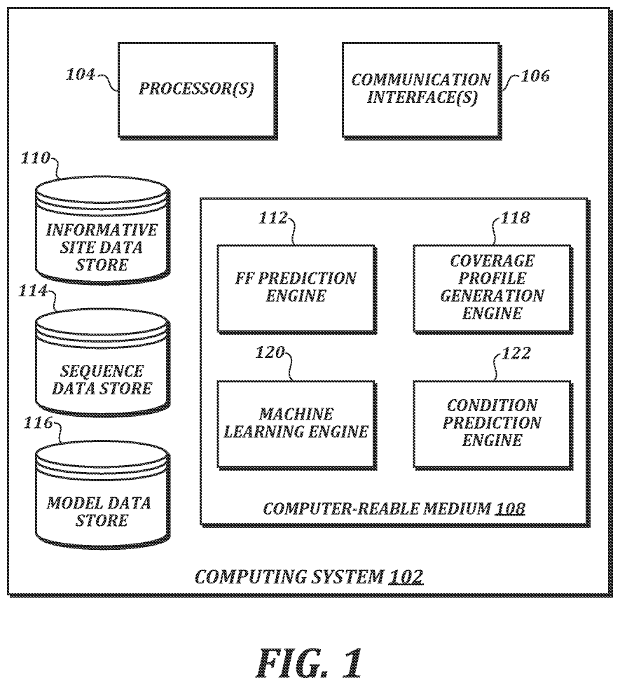

FIG. 1 is a block diagram that illustrates aspects of a non-limiting example embodiment of a computing system configured to estimate FF and/or detect pregnancy-related conditions based on sequence read data, according to various aspects of the present disclosure. The illustrated computing system 102 may be implemented by any computing device or collection of computing devices, including but not limited to a desktop computing device, a laptop computing device, a mobile computing device, a server computing device, a computing device of a cloud computing system, and/or combinations thereof.

As shown, the computing system 102 includes one or more processors 104, one or more communication interfaces 106, an informative site data store 110, a sequence data store 114, a model data store 116, and a computer-readable medium 108.

In some embodiments, the processors 104 may include any suitable type of general-purpose computer processor. In some embodiments, the processors 104 may include one or more special-purpose computer processors or AI accelerators optimized for specific computing tasks, including but not limited to graphical processing units (GPUs), vision processing units (VPTs), and tensor processing units (TPUs).

In some embodiments, the communication interfaces 106 include one or more hardware and or software interfaces suitable for providing communication links between components. The communication interfaces 106 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof.

As shown, the computer-readable medium 108 has stored thereon logic that, in response to execution by the one or more processors 104, cause the computing system 102 to provide a FF prediction engine 112, a coverage profile generation engine 118, a condition prediction engine 122, and a machine learning engine 120.

In some embodiments, the informative site data store 110 may be configured to store identifications of informative sites relevant to determining a FF, relevant to detecting a pregnancy-related condition, or relevant to any other analysis to be performed by the computing system 102. Informative sites are regions of DNA that are accessible based on an absence of nucleosomes. These sites are indicative of a degree and/or presence or absence of a specific biological entity or concept, such as cell type, cell state, subtype, tissue type, etc. These sites may also be determined through hypothesis and/or through statistical testing.

In some embodiments, the sequence data store 114 may be configured to receive and store sequence read data from a sequencing device or instrument. One non-limiting example of a sequencing device is an Illumina NextSeq 500, though any other suitable sequencing device may be used. In some embodiments, the model data store 116 may be configured to store one or more machine learning models trained to determine a FF, to detect a pregnancy-related condition, or to perform any other relevant task based at least on features derived from sequence read data.

In some embodiments, the coverage profile generation engine 118 is configured to retrieve sequence read data stored in the sequence data store 114, retrieve identifications of informative sites from the informative site data store 110 for a given prediction task, and to compute a coverage profile for the informative sites based on the sequence read data. In some embodiments, the FF prediction engine 112 is configured to generate features based on a coverage profile and to provide the features to the machine learning engine 120 to determine a FF for the sequence read data. In some embodiments, the condition prediction engine 122 is configured to generate features based on a coverage profile and to provide the features to the machine learning engine 120 to determine a prediction of a pregnancy-related condition for the sequence read data. In some embodiments, the machine learning engine 120 is configured to train machine learning models to perform the tasks described herein, to store the trained machine learning models in the model data store 116, and to execute the machine learning models to generate predictions based on features received from other components.

Further description of the configuration of each of these components is provided below.

As used herein, “computer-readable medium” refers to a removable or nonremovable device that implements any technology capable of storing information in a volatile or non-volatile manner to be read by a processor of a computing device, including but not limited to: a hard drive; a flash memory; a solid state drive; random-access memory (RAM); read-only memory (ROM); a CD-ROM, a DVD, or other disk storage; a magnetic cassette; a magnetic tape; and a magnetic disk storage.

As used herein, “engine” refers to logic embodied in hardware or software instructions, which can be written in one or more programming languages, including but not limited to C, C++, C#, COBOL, JAVA™, PHP, Perl, HTML, CSS, Javascript, VBScript, ASPX, Go, and Python. An engine may be compiled into executable programs or written in interpreted programming languages. Software engines may be callable from other engines or from themselves. Generally, the engines described herein refer to logical modules that can be merged with other engines, or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device.

As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

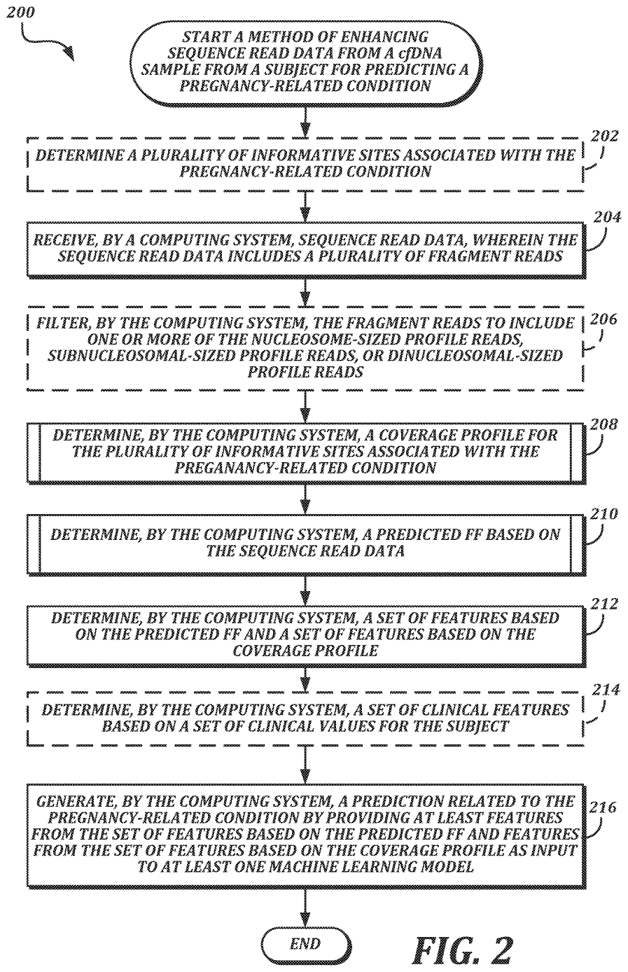

FIG. 2 is a flowchart that illustrates a non-limiting example embodiment of a method of enhancing sequence read data from a cfDNA sample from a subject for predicting a pregnancy-related condition, according to various aspects of the present disclosure. In the method 200, tissue-specific accessibility is analyzed in cfDNA of the subject to measure tissue of origin (including, but not limited to, placental tissue) in order to predict pregnancy-related conditions such as PEP, small neonatal size for gestational age, intrauterine growth restrictions, and/or other conditions at any point in pregnancy, even as early as the first trimester. Accessibility is a state ascribed to a region of DNA that is wound (inaccessible) or not wound (accessible) around nucleosomes. A DNA site deemed “accessible” is available for access by DNA binding proteins including transcription factors and RNA polymerase. In the context of cfDNA, these DNA regions are less protected from degradation by nucleosomes.

From a start block, the method 200 proceeds to optional block 202, where a computing system determines a plurality of informative sites associated with the pregnancy-related condition. In some embodiments, the plurality of informative sites may include at least one open chromatin site specific to a tissue determined to be relevant to the pregnancy-related condition. An open chromatin site, also known as euchromatin, is more lightly packed by nucleosomes than sites that are not open chromatin sites. Open chromatin sites enable accessibility to DNA binding proteins. One root of PEP is placental disfunction, and so placental tissue may be considered relevant to the pregnancy-related condition. Previous work has shown that PEP is characterized by endothelial disfunction. Accordingly, endothelial tissue is another type of tissue that may be considered relevant to the pregnancy-related condition. In some embodiments, other types of tissues may be used, including but not limited to immune cells.

In some embodiments, tissue-specific DNase 1 hypersensitivity sites (DHS) may be obtained, for example from the ENCODE regulatory index, to construct tissue-specific open chromatin maps based on assays such as chromatin immunoprecipitation with sequencing (CHiP-seq). A set of top ChIP-seq coverage peaks that represent the DHS from each tissue (for example, the top 10,000 coverage peaks, or some other threshold number of the top coverage peaks) may be selected based on ranking by their recurrence (i.e., a number of samples) across experiments and mean DHS signal based on the peak coverage. Additionally, a number (e.g., 20, or some other number) of tissue-specific open chromatin maps may be generated from published single-cell transposase-accessible chromatin sequencing (scATAC-seq) data (see Zhang K, Hocker J D, Miller M, Hou X, Chiou J, Poirion O B, Qiu Y, Li Y E, Gaulton K J, Wang A, Preissl S, Ren B. “A single-cell atlas of chromatin accessibility in the human genome.” Cell. 2021 Nov. 24; 184 (24): 5985-6001.e19. doi: 10.1016/j.cell.2021.10.024. Epub 2021 Nov. 12. PMID: 34774128; PMCID: PMC8664161, hereby incorporated by reference herein in its entirety for all purposes.). For open chromatin sites, a set of top peaks (for example, the top 10,000 peaks, or some other threshold number of the top peaks) may be selected from the scATAC-seq data with the highest peak scores based on sequencing coverage analysis using a standard peak calling technique such as MACS2 and considered as informative sites. In some embodiments, informative sites, such as open chromatin sites, may be determined experimentally such as using bulk or single-cell ATAC-seq assays. In one example, a total of n=6 independent placental samples (3 from PEP and 3 from uncomplicated pregnancies) collected through an ongoing study underwent ATAC-seq to identify placental tissue-specific open chromatin sites. In another set of samples, the informative sites from healthy placenta tissue was determined via bulk ATAC-seq library preparation.

In some embodiments, the plurality of informative sites may include at least one transcription factor binding site (TFBS) determined to be relevant to the pregnancy-related condition. In some embodiments, TFBSs may be obtained from the Gene Transcription Regulation Database (GTRD) database, and the top TF-specific binding sites may be filtered based on recurrence across experiments as informative sites.

Once determined, the plurality of informative sites are stored in the informative site data store 110 for later use. The actions of optional block 202 are described as optional because, in some instances of the method 200, the informative sites may have been previously determined and stored in the informative site data store 110, using the same process or another process.

At block 204, the computing system receives sequence read data, wherein the sequence read data includes a plurality of fragment reads. In some embodiments, the sequence read data may be generated by processing samples obtained from a subject. In a non-limiting example embodiment, libraries may be generated using validated, laboratory-developed techniques that have been previously reported. Whole blood may be centrifuged to isolate plasma as per the manufacturer instructions. CfDNA may be extracted from the plasma using standard extraction kits (such as the QIAsymphony Circulating DNA Kit). Following measurement of the DNA concentration extracted, next-generation sequencing library preparation may be performed using materials and equipment such as the BioMek 4000 using the KAPA HyperPrep kit for adapter and index ligation. Libraries may be purified using, for example, the Agencourt AMPureXP kit prior to amplification. Following amplification, the library may be purified on equipment such as the Agilent BRAVO workstation using AMPure beads. Sample pools may be created using an equimolar strategy and diluted down to as low as a lower concentration suitable for a sequencing device, such as 1 nM. Sequencing may then be performed using sequencing instruments, such as an Illumina NextSeq 500, a sequencing device with typical read configuration, such as but not limited to 37 bp paired-end read configuration. The sequence data may be aligned to a reference genome, such as reference genome hg38 (GRCh38). Raw fastq files containing sequence read data may be adapter trimmed using a software tool such as cutadapt tool (v3.3) and then be aligned using an algorithm such as but not limited to BWA-MEM (v0.7.17) to produce a mapped version of the fastq such as a bam file that stores the aligned reads in binary format and further processed using a tool such as SAMtools (v1.10) to sort and index the BAM file. The MarkDuplicates function of Picard tool (v2.18.29) may be used to mark duplicates. The CollectAlignmentSummaryMetrics, CollectWgsMetrics, and CollectInsertSizeMetrics tools functions of the Picard tool may be used to calculate a selection of QC sequencing metrics. In other embodiments, any other suitable technique known to those of skill in the art may be used to generate libraries, sequence cfDNA, and align reads based on the sample. In some embodiments, the sequence read data may be generated as described above, and may then be stored in the sequence data store 114 before being retrieved from the sequence data store 114 by the computing system at block 204. In some embodiments, the sequence read data may be received directly by the computing system from the sequencing device or a computing device performing the alignment.

A fastq file is a text file composed of the DNA sequence content of DNA strands contained in a laboratory-prepared sample containing many DNA molecules, typically following DNA extraction from a source such as human tissue or plasma. A raw fastq file typically contains a set of alphanumeric strings (typically A, C, T, and G), as read by a sequencer (for example an Illumina NextSeq). Typically, each DNA molecule that was read by the sequencer is assigned an identifier and/or description, the sequence letters, and a quality score. Fastq files are typically used as the raw data input to downstream genomics analyses. Fastq files contain the DNA sequences as read from the sample molecules by the sequencer, but may not include any additional contextual information, such as whether the DNA molecules match to any known reference genomes.

A SAM file is a mapped version of a fastq file, typically produced during a sequence alignment to a human (or other species') reference genome. The SAM file typically contains, for each sequenced string of DNA in the fastq file, information such as whether a matching sequence of DNA was found within the chosen reference genome or not, and where applicable the genomic coordinates from within the reference genome pertaining to the sequenced molecule, any discrepancies between the sequenced DNA molecular and the corresponding matched section of DNA in the reference, and a score that proves and assessment of the relative quality of the match between the sample DNA and the section of DNA in the reference genome. A BAM file is a compressed binary representation of the SAM file.

In some embodiments, varying sequencing depths may be used without unduly impacting performance of the method 200. For example, the sequence read data may include data collected at shallow whole genome sequencing depths in the range of 5× down to 0.1× coverage of the whole genome. Other potentially relevant examples of sequencing depths include greater than 5× coverage or lower than 0.1× of the whole genome. The sequencing depth refers to a count of the number of sequenced molecules reported in an aligned sequencing file (for example, a BAM file) at any given position. The sequencing depth is frequently reported as a mean value across the entire reference genome that was used during the sequence alignment, or in some cases to a predefined subset of genomic coordinates.

At optional block 206, the computing system filters the plurality of fragment reads to include one or more of nucleosome-sized profile reads, subnucleosomal-sized profile reads, or dinucleosomal-sized profile reads in order to exclude fragment reads that are unlikely to reflect gene expression. In some embodiments, filtering the fragment reads to include nucleosome-sized profile reads may include filtering the fragment reads to include fragment reads having a size (i.e., a length in base pairs [bp]) in a range from 100 bp to 200 bp (inclusive or exclusive). In some embodiments, filtering the fragment reads to include subnucleosomal-sized profile reads may include filtering the fragment reads to include fragment reads having a size in a range less than 100 bp (inclusive or exclusive). In some embodiments, filtering the fragment reads to include dinucleosome-sized profile reads may include filtering the fragment reads to include fragment reads having a size in a range from 300 bp to 400 bp (inclusive or exclusive). In some embodiments, filtering the fragment reads may use narrower ranges. For example, In some embodiments, filtering the fragment reads to include nucleosome-sized profile reads may include filtering the fragment reads to include fragment reads having a size in a range from 120 bp to 180 bp (inclusive or exclusive). In some embodiments, filtering the fragment reads to include subnucleosomal-sized profile reads may include filtering the fragment reads to include fragment reads having a size in a range from 35 bp to 80 bp (inclusive or exclusive). The optional block 206 is illustrated and described as optional because in some embodiments, such filtering may not be performed, and instead all of the fragment reads received in the sequence read data may be processed.

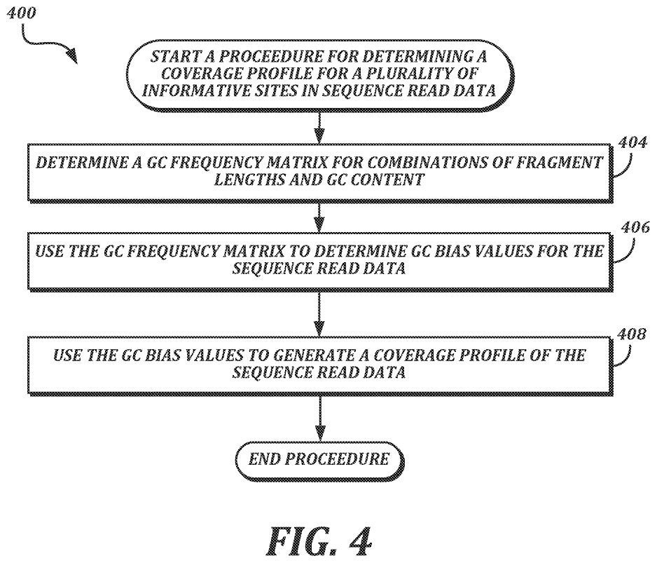

At subroutine block 208, a procedure is executed wherein the computing system determines a coverage profile for the plurality of informative sites associated with the pregnancy-related condition. In some embodiments, the method 200 may provide the informative sites from optional block 202 to the procedure that determines the coverage profile, along with the plurality of fragment reads. In some embodiments, the coverage profile may be determined for windows of a predetermined size flanking each informative site. For example, in some embodiments, the coverage profile may be determined for base pairs in a window, for example from 1000 bp before each informative site to 1000 bp after each informative site (i.e., ±1000 bp from each informative site). Other window size ranges are also possible, such as window sizes in a range from ±500 bp to ±5,000 bp. Any suitable technique may be used to generate the coverage profile. In some embodiments, techniques that compensate for GC bias, referred to as “Griffin,” may be used to generate the coverage profile. One non-limiting example embodiment of such a technique for generating the coverage profile that compensates for GC bias is illustrated in FIG. 4 and described in further detail below.

At subroutine block 210, the computing system determines a predicted FF based on the sequence read data. The FF represents an amount of cfDNA in the sample that derives from the fetal/placental unit. In some embodiments, the FF may represent a relative amount of fetal/placental DNA in the sample, such as a percentage of the cfDNA that is fetal/placental DNA versus maternal DNA, or a percentage of the cfDNA that is fetal/placental DNA versus all cfDNA (e.g., identifiable maternal DNA, fetal/placental DNA, and unidentifiable DNA), or any other quantification of the fetal/placental DNA with respect to any quantification of the sample as a whole.

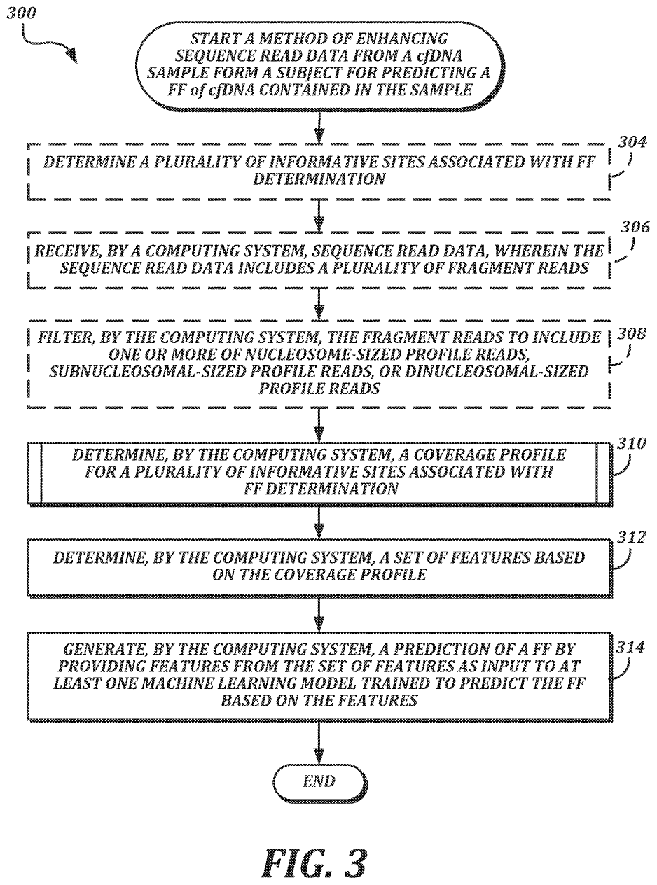

Any suitable technique may be used to determine the predicted FF based on the sequence read data. In some embodiments, features extracted from a coverage profile for either the same informative sites as determined in optional block 202 or different informative sites may be used to predict the FF using a machine learning model trained to predict FF. A non-limiting example embodiment of such a technique is illustrated in FIG. 3 and discussed in further detail below.

In some embodiments in which the fetus is known to be male, a chromosome Y-based FF (ChrY-FF) calculation may be used. The ChrY-FF may be calculated as the following formulas:

$$ChrY \% = \frac{\sum ( Reads mapped to Chromosome Y )}{\sum ( All reads )}$$

where Σ(Reads mapped to Chromosome Y) refers to the number of fragment sequence reads for a given sample that align to Chromosome Y and Σ(All reads) refers to the number of reads for the same sample that align to the reference genome.

To adjust for erroneously aligning reads to ChrY, a baseline set of male samples and samples from pregnant women with known female fetuses may be used to estimate average percent reads mapping to ChrY:

$$Average male ChrY \% = \frac{\sum ( Male ChrY \% )}{n}$$

where Σ(Male ChrY %) refers to the sum of the individual ChrY % values obtained for each sample in the baseline set of known male samples and n refers to the number of male samples contained in the set; and

$$Average female ChrY \% = \frac{\sum ( Female ChrY \% )}{n}$$

where Σ(Female ChrY %) refers to the sum of the individual ChrY % values obtained for each sample in the set of baseline samples from pregnant women with known female fetuses and n refers to the number of samples from pregnant women with known female fetuses contained in the set.

Finally, the ChrY-FF may be calculated by adjusting for the average erroneously mapped reads:

$$ChrY - FF = \frac{( ChrY \% - Average female ChrY \% )}{( Average male ChrY \% - Average female ChrY \% )}$$

At block 212, the computing system determines a set of features based on the predicted FF and a set of features based on the coverage profile. For the predicted FF, one appropriate feature may be the predicted FF value itself. For the set of features based on the coverage profile, any suitable features may be extracted. One suitable feature is a nucleosome-depleted region (NDR) score. The NDR score may be determined as the mean coverage value from the coverage profile of a region having a size in a range of +1 bp to #250 bp, for example+30 bp, from each informative site (e.g., a summit or a transcription factor binding site). Another suitable feature is a mean coverage value (MCV) score. The MCV score may be determined as the mean coverage value in a region having a size in a range of +250 bp to +5000 bp, for example+1000 bp, from each informative site. Other sizes of regions are possible for NDR scores and MCV scores.

At optional block 214, the computing system determines a set of clinical features based on a set of clinical values for the subject. In some embodiments of the method 200, additional predictive value may be obtained by collecting features other than the coverage profile features. For some pregnancy-related conditions such as PEP, there are recognized clinical factors that are considered correlated with or signs of risk for developing the pregnancy-related condition, and so clinical features based on clinical values related to those factors may add predictive value to the method 200. One non-limiting example of such clinical factors is blood pressure as indicated by one or more blood pressure measurement values, such as a systolic blood pressure value (SBP) or a diastolic blood pressure (DBP) value. Another non-limiting example of such clinical factors is body mass index (BMI) value. The computing system may receive clinical values such as the SBP, DBP, or BMI values from an electronic medical record system, from user input, or in any other suitable way. The determined set of clinical features may be the clinical values themselves or any other suitable representation based on the clinical values. The optional block 214 is illustrated and described as optional because in some embodiments, the coverage profile features may be used for prediction without the clinical features.

At block 216, the computing system generates a prediction related to the pregnancy-related condition by providing at least features from the set of features based on the predicted FF and features from the set of features based on the coverage profile as input to at least one machine learning model. In some embodiments, multiple machine learning models may be trained to accept different features as input, with the output being analyzed as an ensemble to produce a final result. In some embodiments, a first machine learning model may be trained to accept the set of features based on the coverage profile and the set of features based on the predicted FF as input, a second machine learning model may be trained to accept the set of clinical features as input, and a third machine learning model may be trained to accept the set of features based on the coverage profile, the set of features based on the predicted FF, and the set of clinical features as input. Output of each machine learning model may be added to each other, averaged with each other, or combined in any other suitable way to generate an ensemble prediction.

In some embodiments, the output represents a probability that the subject will experience the pregnancy-related condition. In some embodiments, the output may also reflect a predicted severity of the pregnancy-related condition (e.g., higher output values may represent a more severe version of the pregnancy-related condition). As a non-limiting example, for PEP, the output may indicate a likelihood of a normal pregnancy, early-onset PEP, late-onset PEP, or late-onset PEP with pre-term birth. Though PEP is mentioned above, in other embodiments, the output represents a probability of a different pregnancy-related condition, including but not limited to small neonatal size for gestational age or intrauterine growth restrictions.

Any suitable architecture may be used for the one or more machine learning models. As non-limiting examples, a logistic regression model (e.g., an L1-normalized logistic regression model, an L2-normalized logistic regression model, or other types of logistic regression models) may be used. One specific non-limiting example of a suitable logistic regression model is an sklearn LogisticRegression model with class weight balanced, v1.1.1, though others may be used. As another non-limiting example, an XGBoost model may be used. One specific non-limiting example of an XGBoost model is an XGBoost XGBClassifier, with a number of estimators set to 50, and a max depth of 10, v1.6.1, though others may be used and other settings for the hyperparameters may be used. Other examples of suitable machine learning models include, but are not limited to, random forest models, probabilistic models, latent state probabilistic models, Bayesian statistical models, and generative probabilistic models. The machine learning models may be trained using any suitable technique or combination of techniques, including but not limited to gradient descent and/or bootstrapping with replacement.

Once the output is generated, the output may be provided to an operator as the prediction of the pregnancy-related condition (and, optionally, its predicted severity), may be stored for later use, may be used to automatically control a treatment device, or may be used for any other purpose. The method 200 then proceeds to an end block and terminates.

FIG. 3 is a flowchart that illustrates a non-limiting example embodiment of a method for enhancing sequence read data from a cfDNA sample from a subject for predicting a FF of cfDNA contained in the sample, according to various aspects of the present disclosure. In the method 300, tissue-specific accessibility is analyzed in cfDNA of the subject to predict the FF of the cfDNA. The method 300 is a non-limiting example of a technique suitable for use at subroutine block 210 of FIG. 2, but may also be used separately from the method 200 to predict the FF to be used for other purposes.

From a start block, the method 300 advances to optional block 304, wherein a computing system determines a plurality of informative sites associated with FF determination. Placenta and immune cell tissues have been identified to be both negatively and positively correlated with ground truth FF values. A ground truth is a value or set of values determined through a trusted methodology to be used as a reference against with an experimentally derived value or set of values may be compared. Accordingly, in some embodiments, the plurality of informative sites may be associated with placenta and/or immune cell tissues. In other embodiments, additional or other tissues predictive of FF may be used. The plurality of informative sites may include at least one open chromatin site associated with a tissue predictive of FF, and/or at least one TFBS associated with a tissue predictive of FF. As one non-limiting example, Grainy head-like 2 (GHRL2) transcription factor (TF) is known to be highly expressed in the trophoblast cells of placental tissue where it plays a crucial role in placental morphogenesis. Accordingly, TFBSs associated with GHRL2 may be included in the plurality of informative sites. Other TFBSs to be included in the plurality of informative sites may include, but are not limited to, one or more of binding sites for a TEAD4 TF, a TEAD1 TF, a GATA3 TF, or a TFAP2A TF, which have been found to be differentially expressed in placental tissue. Blood-specific TFs, including but not limited to Lymphoblastic leukemia 1 (LYL1) TF, MECOM TF, RUNX1 TF, and NR4A1 TF have been found to have a negative correlation, and so one or more associated TFBSs may also be considered for including as informative sites.

Once determined, the plurality of informative sites are stored in the informative site data store 110 for later use. The actions of optional block 304 are described as optional because, in some instances of the method 300, the informative sites may have been previously determined and stored in the informative site data store 110, using the same process (or another process).

At optional block 306, a computing system receives sequence read data, wherein the sequence read data includes a plurality of fragment reads. The sequence read data may be obtained and received using a technique similar to that described above in block 204, and so the details of obtaining and receiving the sequence read data are not repeated here for the sake of brevity. The optional block 306 is illustrated as optional because, in some embodiments, the sequence read data received at block 204 may be reused by the method 300, and so the sequence read data may not need to be received again. Accordingly, in some embodiments, the method 300 may receive the sequence read data from the sequence data store 114.

At optional block 308, the computing system filters the fragment reads to include one or more of nucleosome-sized profile reads, subnucleosomal-sized profile reads, or dinucleosomal-sized profile reads. Similar filtering was described above in optional block 206, and so the details are omitted here for the sake of brevity. The optional block 308 is illustrated as optional because in some embodiments, such filtering may not be performed, or may have been performed prior to the start of method 300 (such as if the filtered sequence read data from method 200 is being reused).

At subroutine block 310, a procedure is executed wherein the computing system determines a coverage profile for a plurality of informative sites associated with FF determination. In some embodiments, the method 300 may provide the informative sites from optional block 304 to the procedure that determines the coverage profile, along with the plurality of fragment reads. Similar to the determination of the coverage profile at subroutine block 208, subroutine block 310 may use a technique such as Griffin to generate the coverage profile, as illustrated in FIG. 4 and described in further detail below.

At block 312, the computing system determines a set of features based on the coverage profile. As with the set of features based on the coverage profile determined in block 212, the set of features based on the coverage profile determined at block 312 may include any suitable features, including but not limited to NDR scores and/or MCV scores.

At block 314, the computing system generates a prediction of a FF by providing features from the set of features as input to at least one machine learning model. Any suitable machine learning model may be used as the one or more machine learning model. In some embodiments, a non-limiting example of a regularized linear regression model may be used. In some embodiments, another non-limiting example of a Bayesian Ridge regression framework, such as sklearn BayesianRidge, v1.1.1, may be used. In some embodiments, the model may be trained by gradient descent (or any other suitable technique) using a different measurement of FF, such as a determined ChrY-FF value, as the ground truth. In some embodiments, the output of the at least one machine learning model may be a continuous value that represents the FF of cfDNA in the sample.

The method 300 then advances to an end block and terminates, returning control to its caller.

FIG. 4 is a flowchart that illustrates a non-limiting example embodiment of a procedure for determining a coverage profile for a plurality of informative sites in sequence read data according to various aspects of the present disclosure. The procedure 400, described previously as embodying a technique referred to as “Griffin,” generates a coverage profile that is corrected for GC bias.

From a start block, the procedure 400 advances to block 404, where a computing system determines a GC frequency matrix for combinations of fragment lengths and GC content. For certain sequencing technologies, fragments having certain amounts of G and C bases (“GC content”) will be overrepresented in the sequence read data. This bias is not constant, as fragments of different sizes will have different GC biases. Because sequence read data from cfDNA fragments typically includes short fragments of many different lengths, establishing a GC frequency matrix that specifies expected proportions of GC content for various different fragment lengths allows sequence read data to be properly corrected for the GC bias, and for meaningful signals to be obtained from sequence read data that would otherwise be too noisy.

Any suitable technique may be used to determine the GC frequency matrix. In some embodiments, to determine the GC frequency matrix, all mappable regions of the genome are examined. Then, for each fragment length, a number of times each GC content is observed within fragments of the fragment length in the mappable regions is counted to determine GC frequencies for the genome. The GC frequencies are then stored in the GC frequency matrix for the fragment length.

In some embodiments, a range of fragment lengths between a short length threshold and a long length threshold are analyzed to create the GC frequency matrix. In some embodiments, the short length threshold may be in a range of 10-20 bp, and the long length threshold may be in a range of 450-550 bp. In one particular non-limiting example embodiment, the short length threshold may be 15 bp, and the long length threshold may be 500 bp.

At block 406, the computing system uses the GC frequency matrix to determine GC bias values for the sequence read data. Any suitable technique may be used to determine the GC bias values for the sequence read data. In some embodiments, the number of observed reads of each fragment length and GC content are counted to determine GC counts for the sequence read data. The GC counts are divided by the values in the GC frequency matrix to determine GC bias for each fragment length. A mean GC bias is normalized for each fragment length to determine rough GC bias values. In some embodiments, the mean GC bias may be normalized to 1. This results in a rough GC bias value for every possible combination of fragment size and GC content. The rough GC bias values are then smoothed to determine the GC bias values. In some embodiments, for each fragment size, all GC bias values for similar sized fragments (as a non-limiting example, for 165 bp fragments, fragments of sizes from 155 bp to 175 bp may be considered) may be determined. The GC bias values for the similar sized fragments may be sorted by GC content, and kernel smoothing techniques may be performed by taking the median of the nearest neighbors to determine the GC bias values.

At block 408, the computing system uses the GC bias values to generate a coverage profile of the sequence read data for the informative sites. Any suitable technique may be used to generate the coverage profile. In some embodiments, fragment midpoints in a window around each cell-type-specific informative site are determined. A weight is assigned to each fragment based on the appropriate GC bias value for the fragment length and GC content (i.e., the GC bias value for the fragment length and GC content determined at block 406). The weight is then based on that appropriate GC bias value. In some embodiments, the weight may be the inverse of the GC bias value (1/GC bias value). For instance, if 165 bp fragments with 60% GC content have a GC bias of 2.5 in a given sample (overrepresented relative to 165 bp fragments with other GC contents), a weight of 1/2.5=0.4 would be assigned to these fragments. The weights are used to determine GC-corrected midpoint profiles. Positions are excluded that overlap excluded regions. The excluded regions may be determined using any suitable technique. In some embodiments, the excluded regions may be obtained from one or more excluded region lists. Excluded region lists may include, but are not limited to, an encode unified GRCh38 exclusion list, centromeres, gaps in the human genome assembly, fix patches, alternative haplotypes, regions having zero mappability score (e.g. sequence in region contains more than 10 matches in the genome), and regions with high coverage (for example, 10 standard deviations above the mean).

Next, GC-corrected midpoint profiles for all sites are averaged to determine a mean profile, and the mean profile is smoothed to generate a smoothed mean profile. Any suitable technique for smoothing may be used. For example, in some embodiments, the mean profile may be smoothed using a Savitzky-Golay filter with a window length of 165 bp and a 3rd order polynomial. The smoothed mean profile is normalized by dividing by the mean of the surrounding coverage. In some embodiments, surrounding coverage in a range of 9,000-11,000 bp (+/−4,500 bp to +/−5,500 bp), such as 10,000 bp (+/−5,000 bp) is considered for normalization. This allows samples with different depths of sequencing coverage to be compared. The normalized mean profile may then be provided as the resulting coverage profile.

The procedure 400 then advances to an end block and terminates, returning control to its caller along with the coverage profile as a result.

### Example Embodiments and Test Results

First, a framework was developed to estimate the FF and risk of PEP in clinical NIPS sequence read data from a cohort of 1,565 pregnant individuals. Clinical NIPS plasma cfDNA sequence read data was obtained for 1,417 pregnant individuals from the University of Washington. These samples were collected predominantly during the first trimester (n=1043), and the remaining during the second (n=287) and third (n=67) trimesters. An additional 20 samples from non-pregnant females were sequenced through the same NIPS assay. Pregnancy outcomes were determined for 960 samples by clinician review, including PEP and whether the neonate was small for gestational age. For the purpose of this example, the focus was primarily on samples that were sequenced early in gestational age (<=16 weeks gestation, n=755, mean gestational age at sample collection=12.0 weeks). An additional 261 NIPS plasma cfDNA samples with known pregnancy outcome were obtained as a validation cohort.

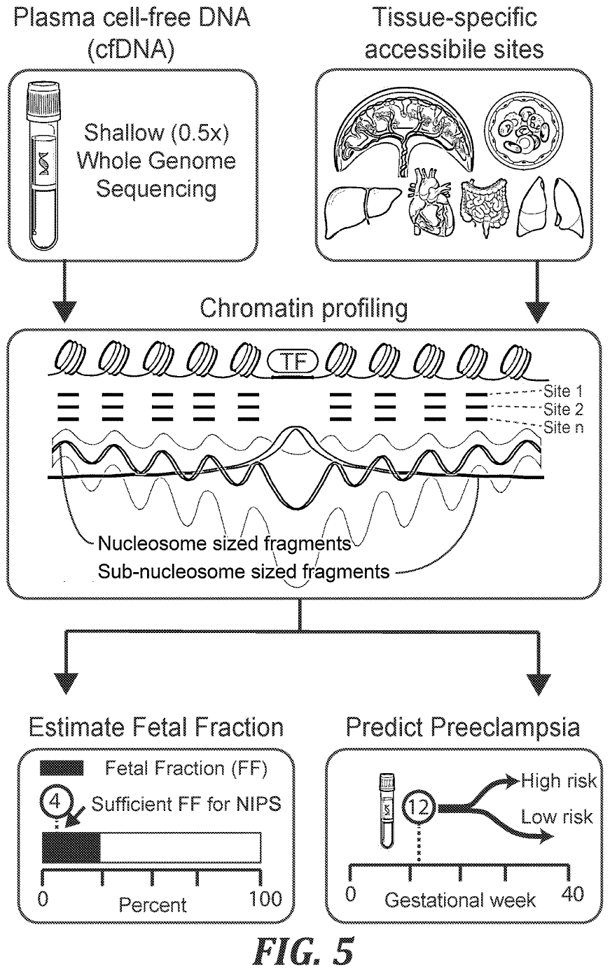

A framework (FIG. 5) was developed to analyze NIPS data, which includes shallow WGS (sWGS) of plasma cfDNA at shallow sequencing depths (mean 0.54, range 0.10-2.91). First, Griffin is applied (e.g., procedure 400 illustrated in FIG. 4) to assess the nucleosome positioning in cfDNA by aggregating across known tissue-specific open chromatin sites into composite profiles. These composite profiles allow shared nucleosome accessibility signals from multiple sites to be detected despite the shallow coverage sequencing depths. To measure nucleosome accessibility, features corresponding to the nucleosome depleted region (NDR, ±30 bp) and mean coverage of the site (MCV, ±1,000 bp) are extracted from the nucleosome profiles. Finally, these features inform the tissues-of-origin, which were used as features to develop machine learning models for (1) improving the estimation of the FF and (2) devising a new approach to predict the risk of PEP.

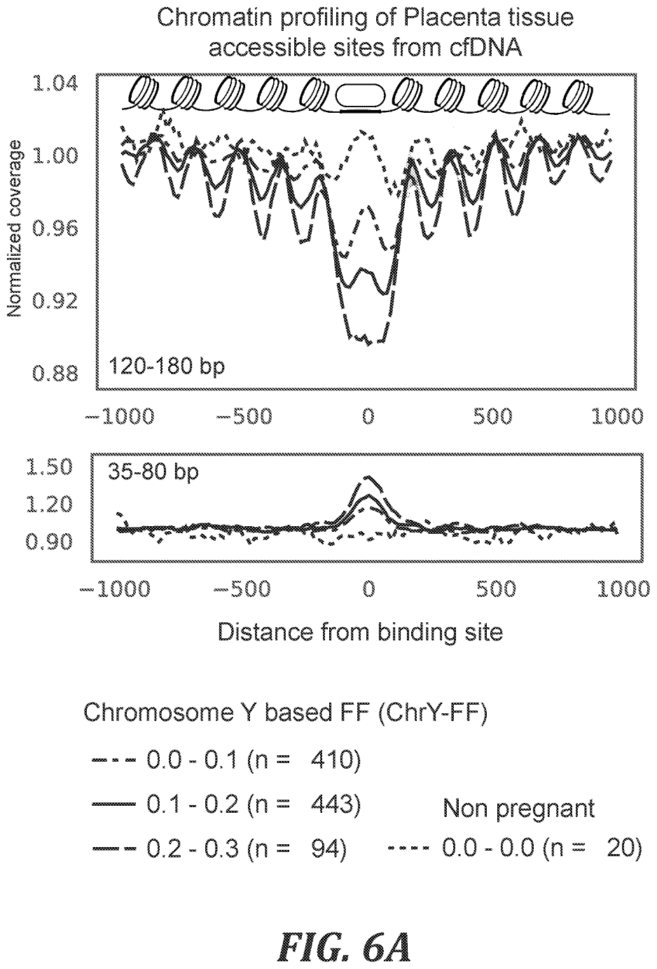

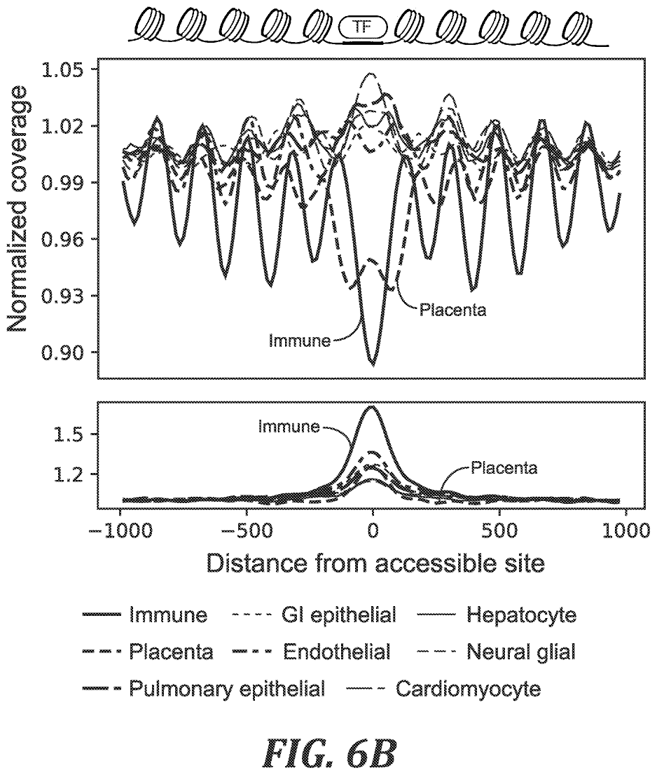

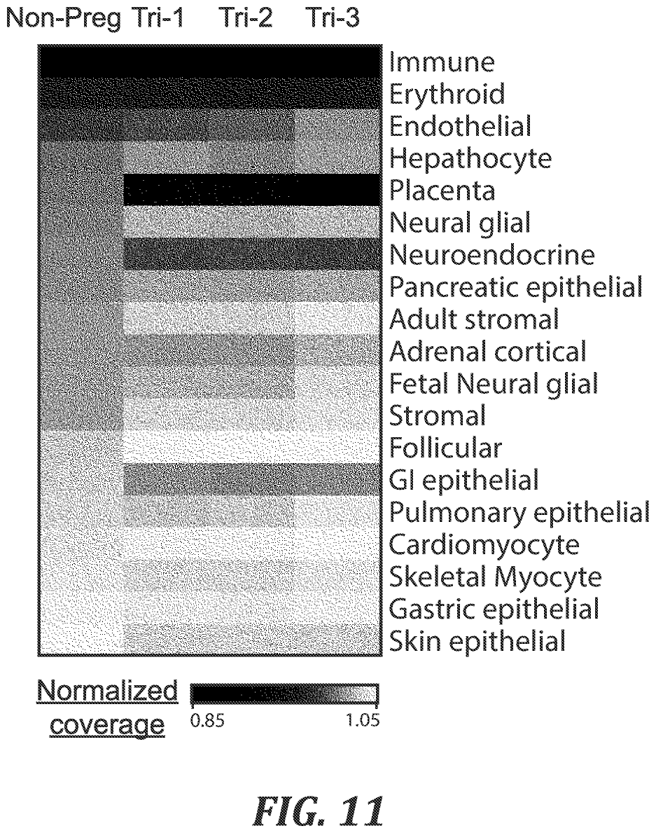

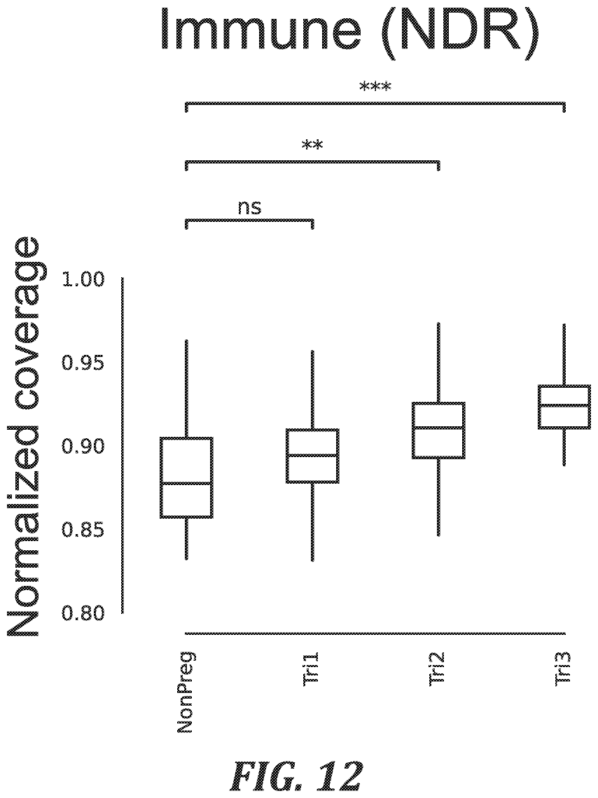

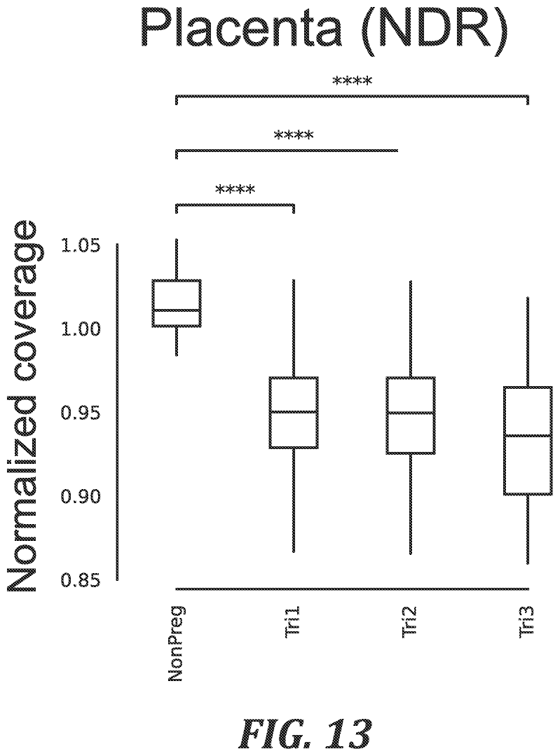

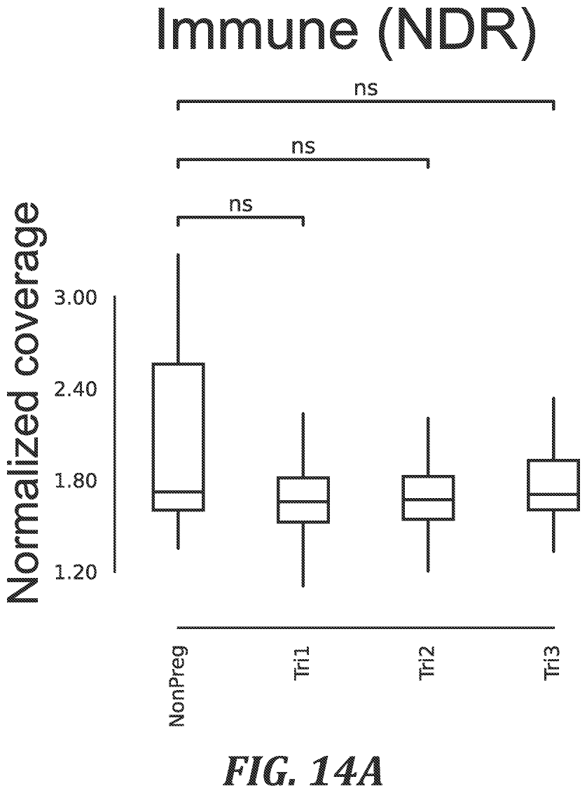

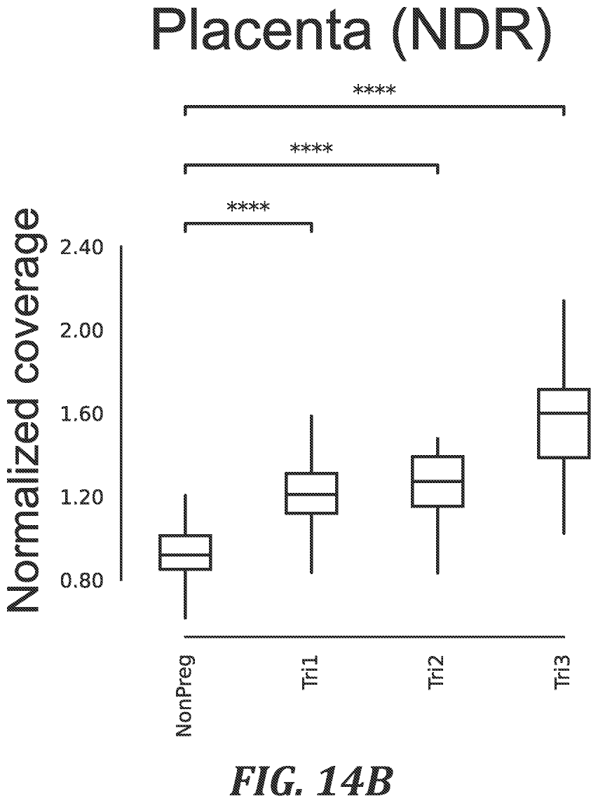

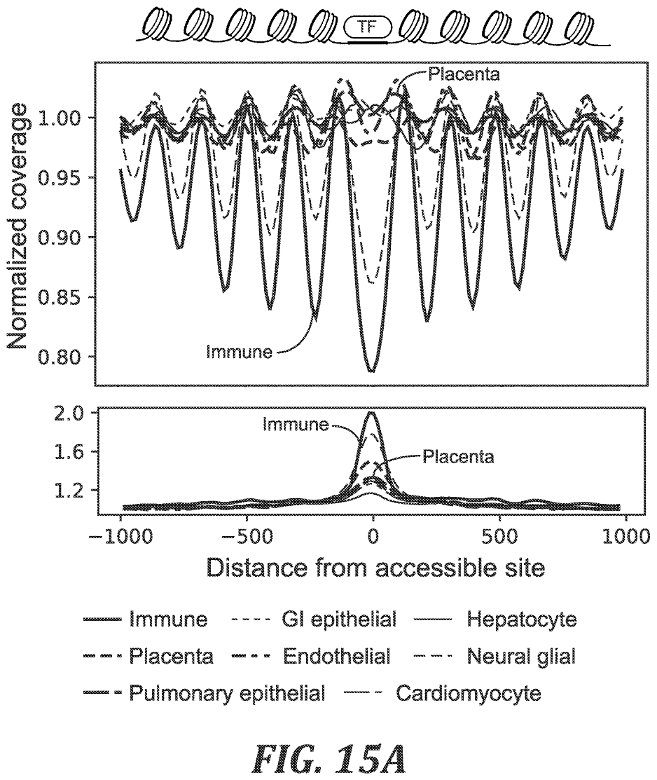

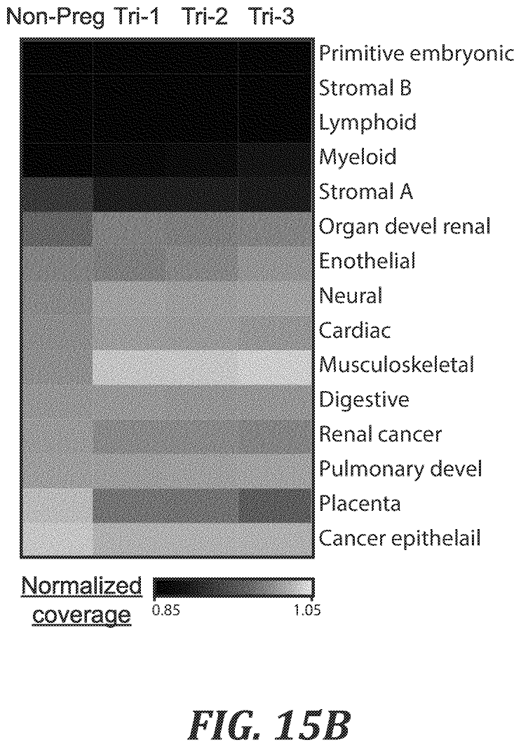

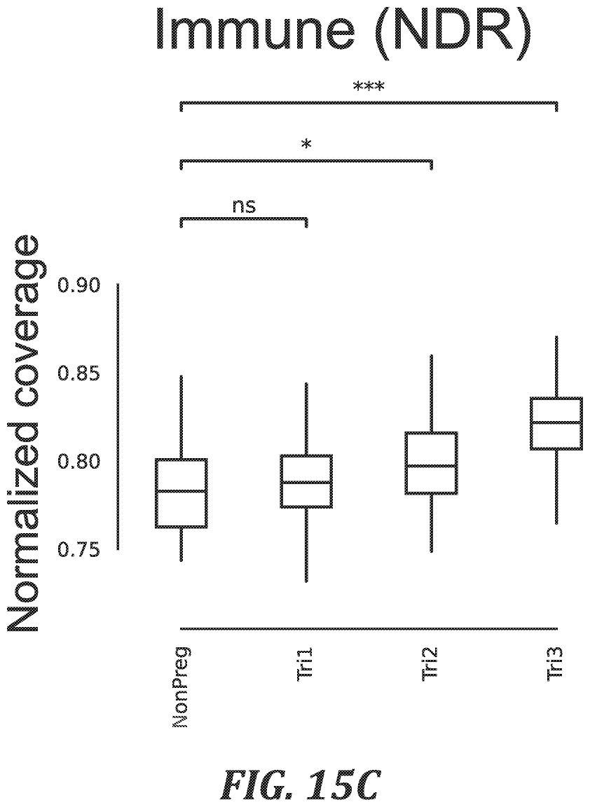

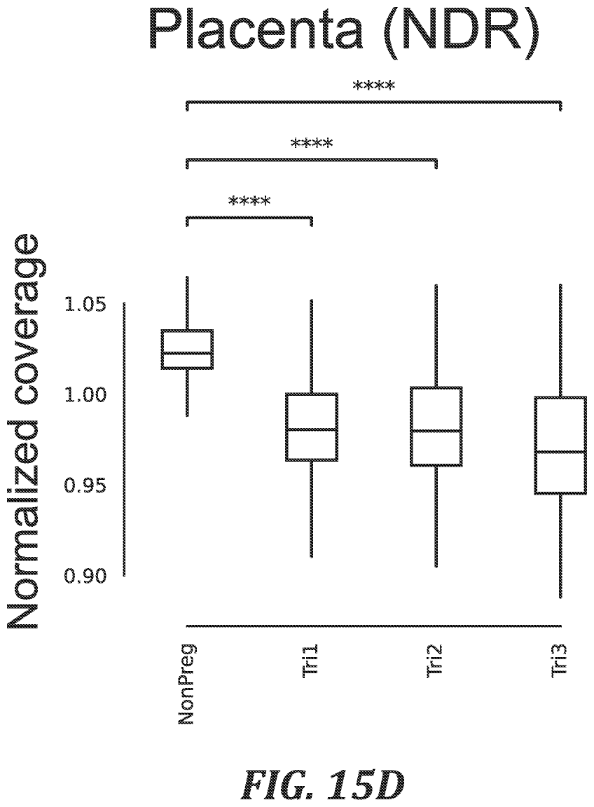

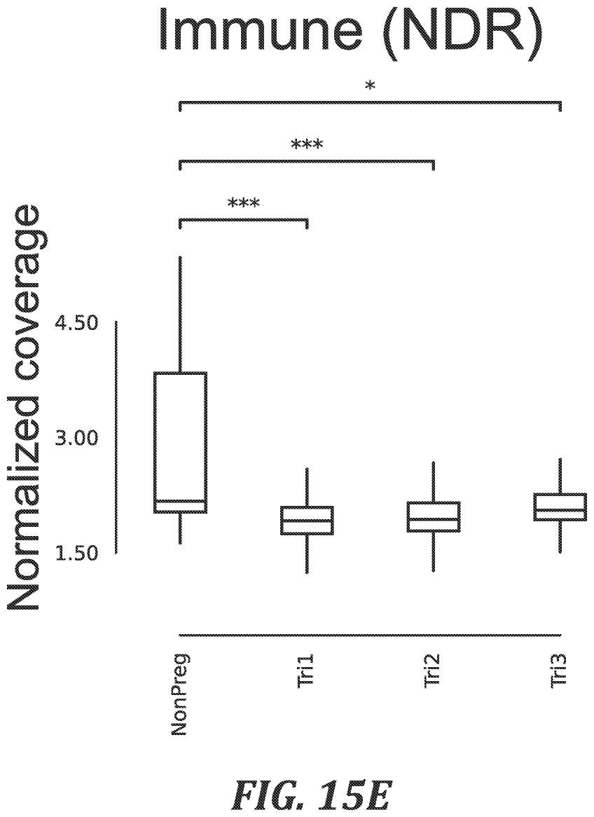

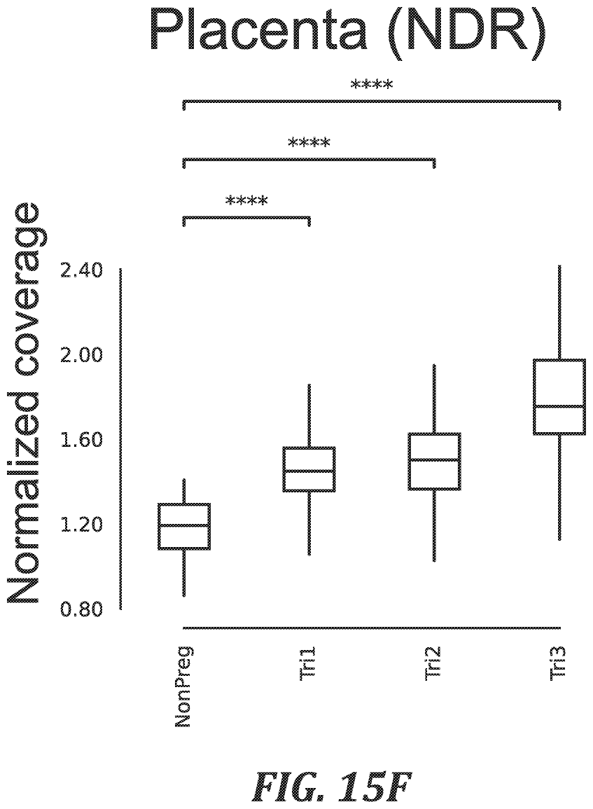

Whether or not accessible chromatin regions of placenta tissue are discernible in maternal cfDNA was also investigated. To determine if nucleosome profiles from sWGS (sWGS 0.5×) of cfDNA can specifically discern its origin from the placenta and other tissues, tissue-specific chromatin accessibility sites derived from publicly available scATAC-seq experiments generated for fetal and adult tissue types were curated. For each tissue type, nucleosome profiles were characterized at accessible sites in the training cohort of 1418 cfDNA samples and observed that immune cells and placental cells had the strongest signals of accessibility at the nucleosome depleted region (NDR) as indicated by decrease in fragment coverage (FIG. 6A, FIG. 6B). The accessibility at placenta tissue sites in NIPS samples increased by trimester while in 20 non-pregnant donor samples had significantly lower accessibility (Mann Whitney U Test, P-value <1.1e-10) (FIG. 13). Conversely, the accessibility at sites associated with immune cells were strongest for non-pregnant women and decreased by trimesters in NIPS samples (Mann Whitney U Test, P-value <5.8e-2) (FIG. 12). A similar trend in the accessibility of sites specific to erythroid and endothelial cells was observed, collectively supporting that the majority of cfDNA originated from hematopoietic cell types (FIG. 11). When considering various cfDNA fragment sizes, an inverse trend was observed for sub-nucleosome (35-80 bp) fragments (FIG. 14A, FIG. 14B). CfDNA was also analyzed for an additional 16 sets of tissues derived from DNase I hypersensitivity sites sequencing (DNase-seq) and observed similarly strong accessibility trends in immune and placenta tissue types (FIG. 15A-FIG. 15F). Altogether, these results demonstrate that evaluating the cfDNA nucleosome accessibility can detect the placenta, hematopoietic, and other tissues-of-origin in NIPS plasma samples.

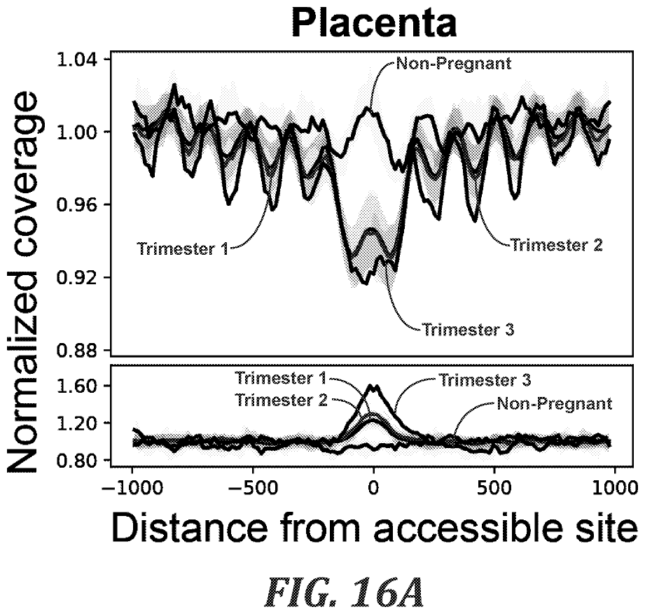

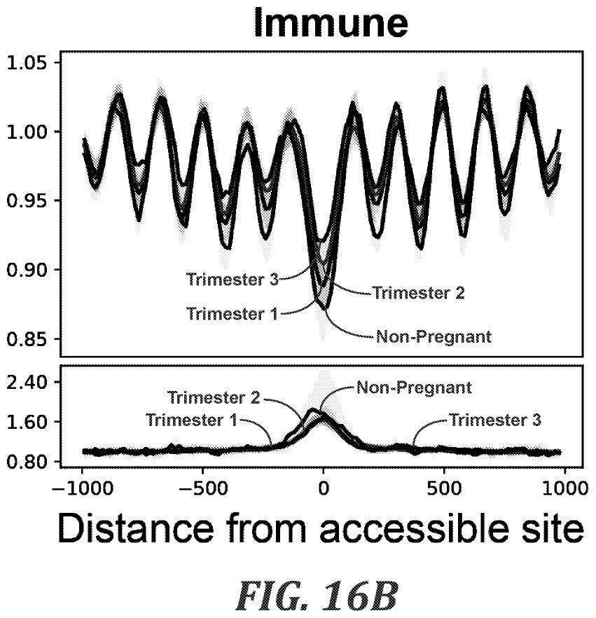

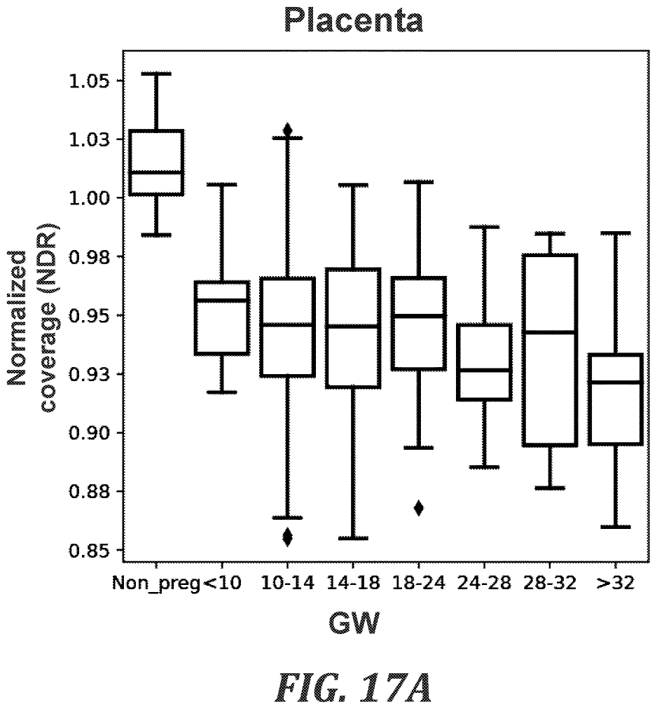

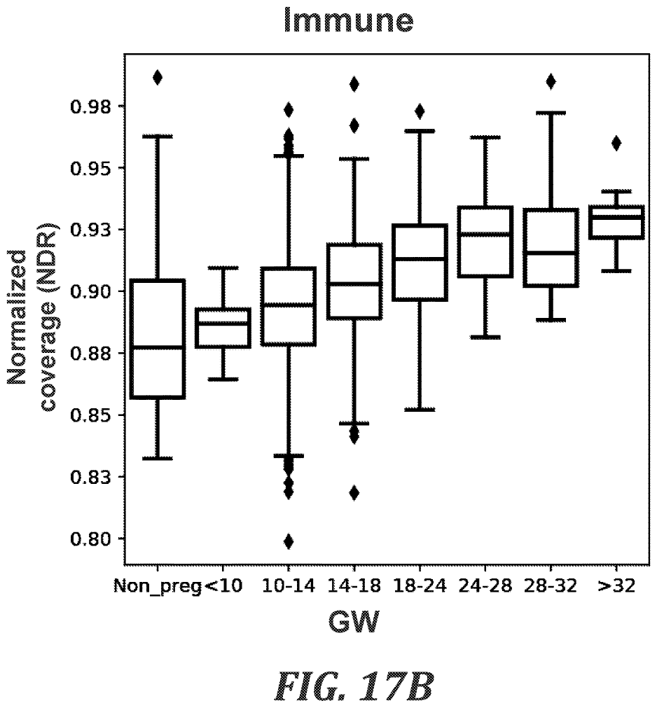

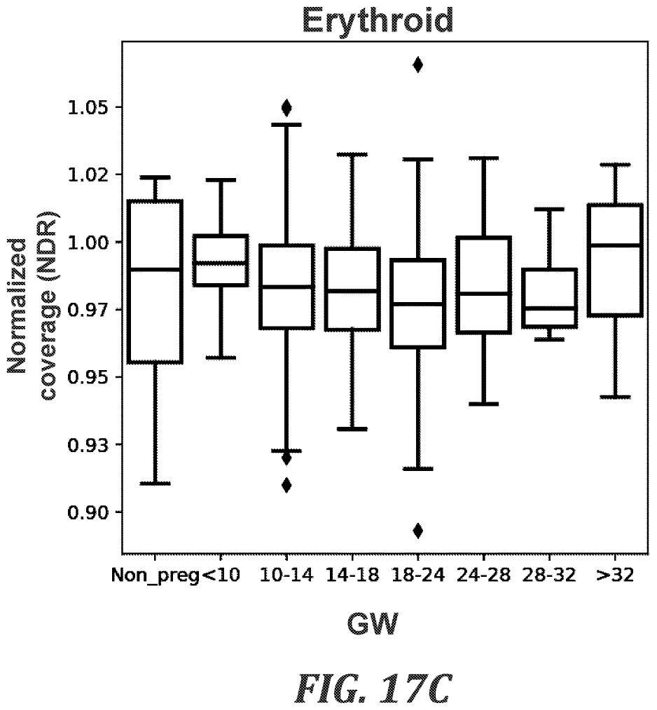

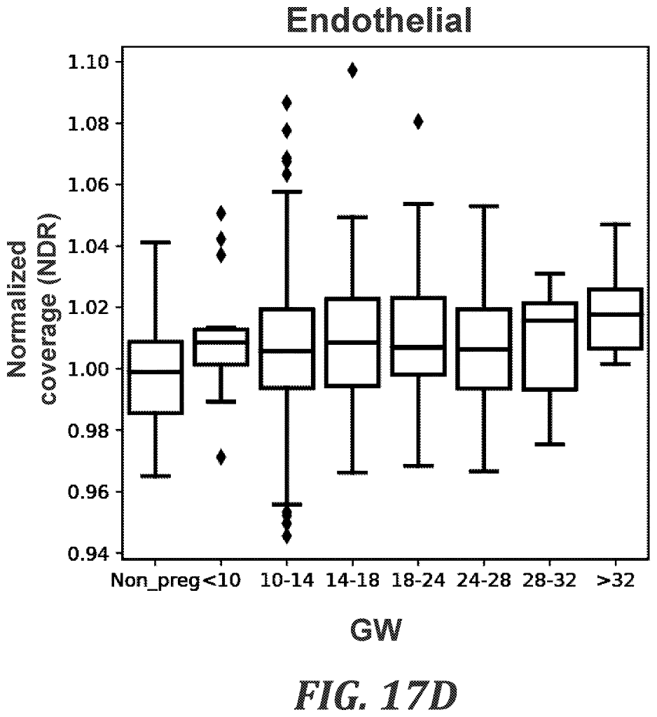

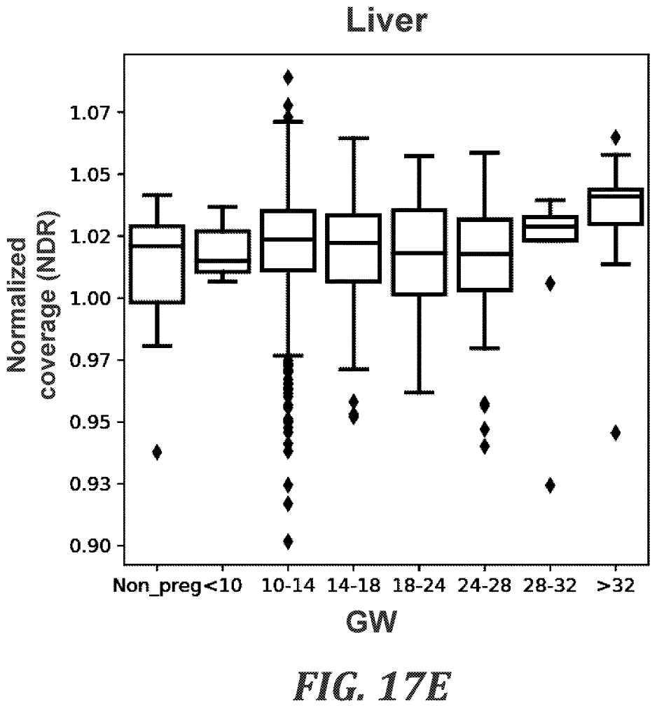

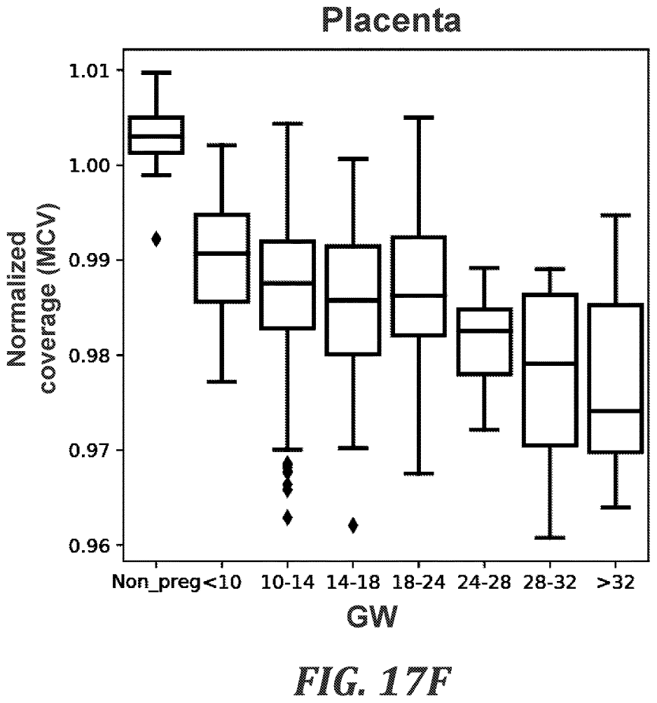

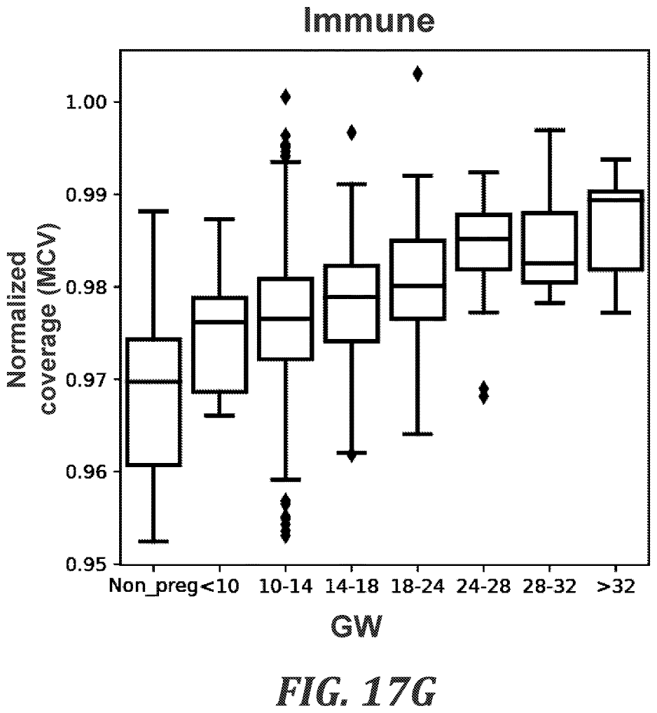

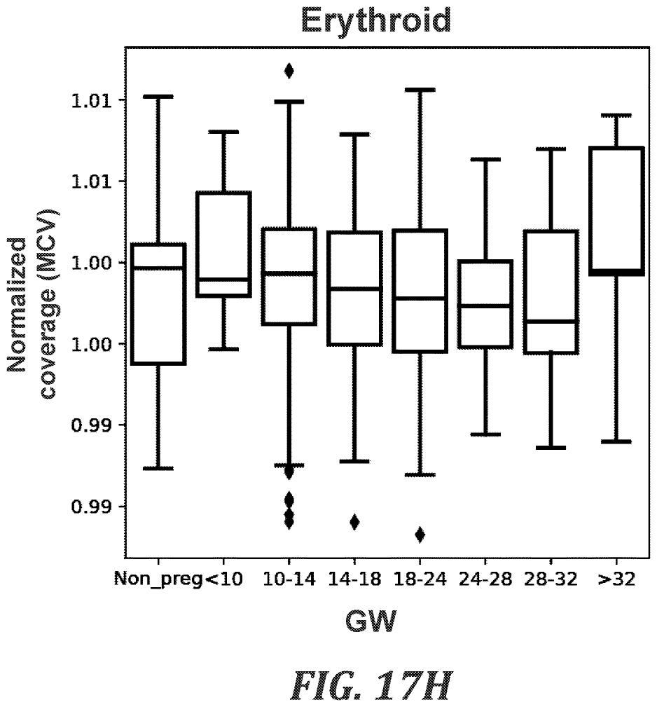

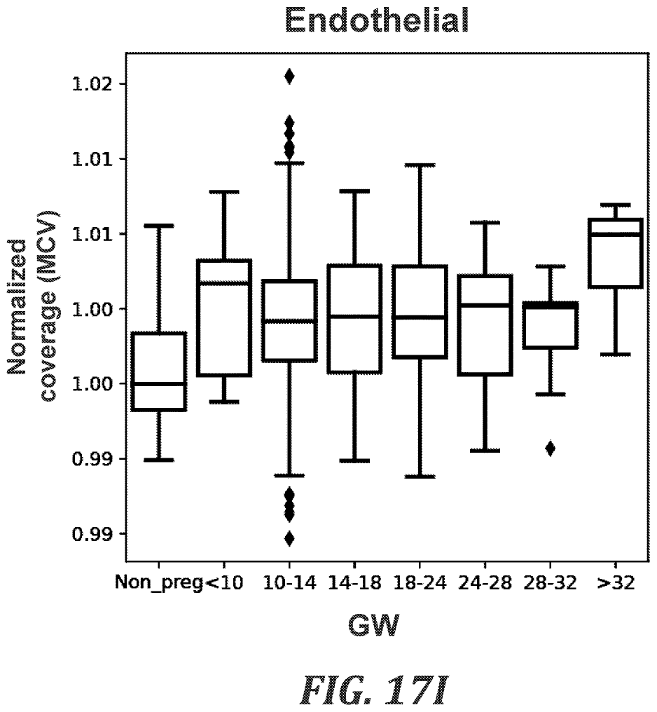

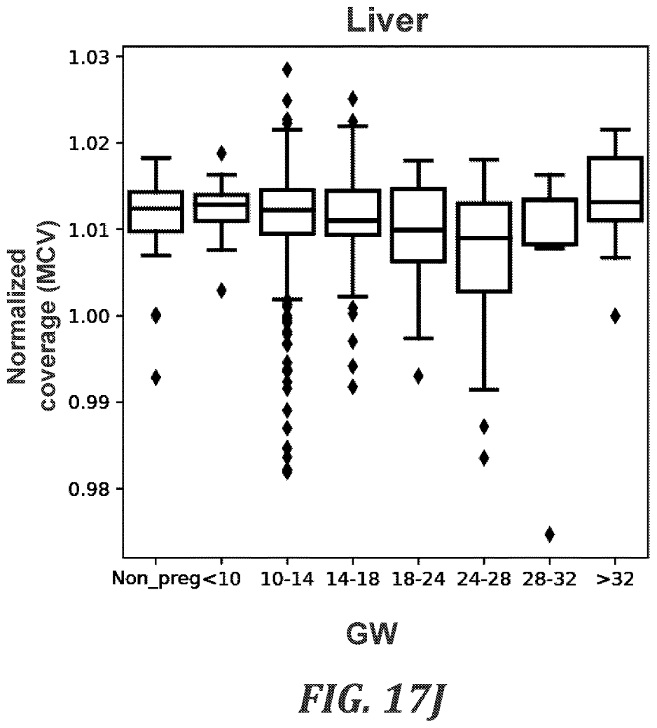

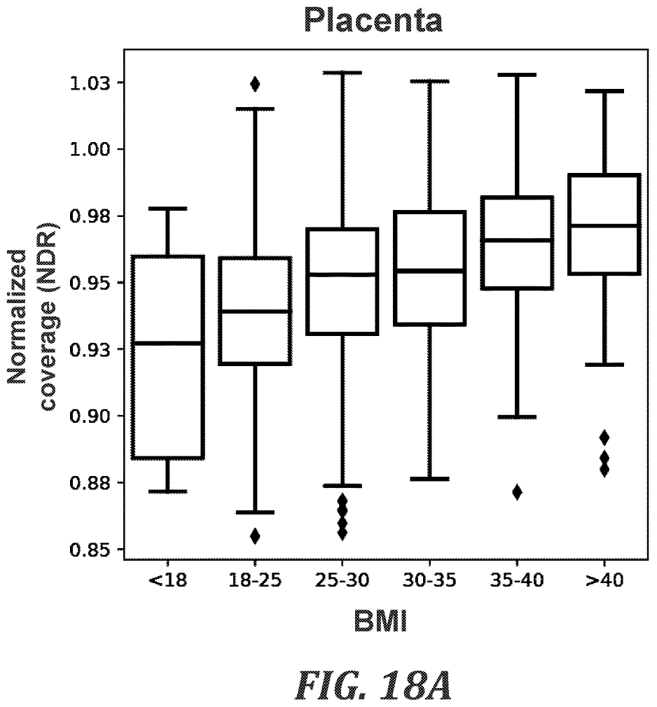

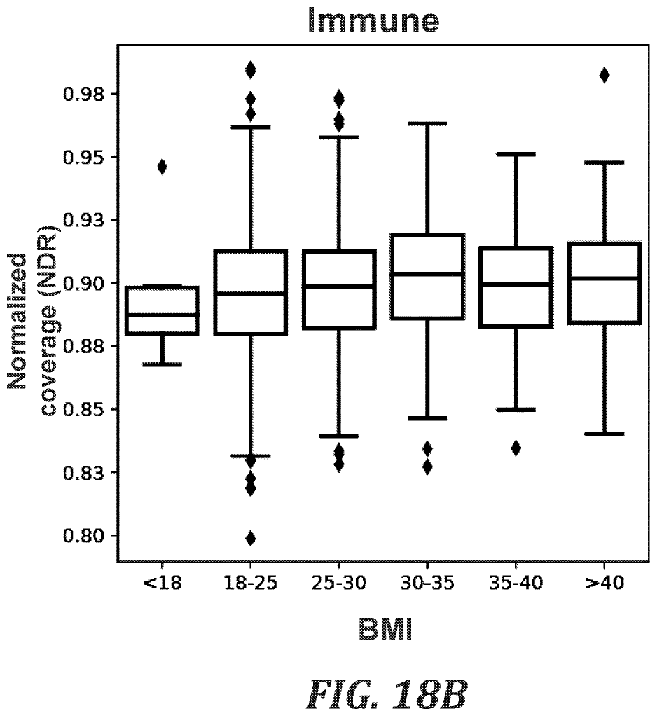

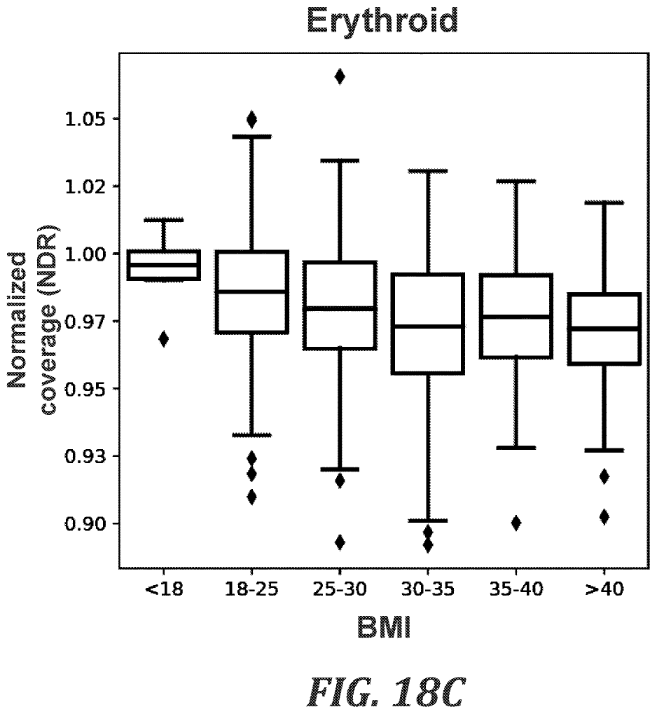

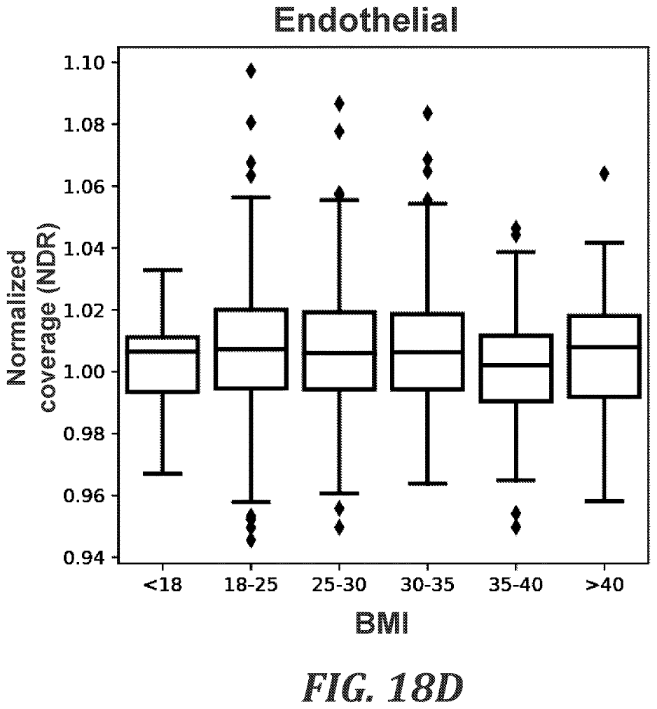

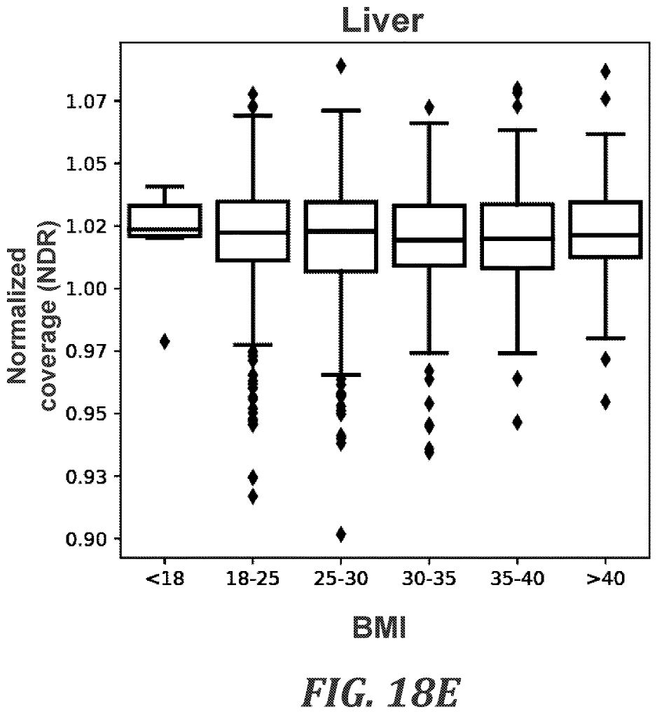

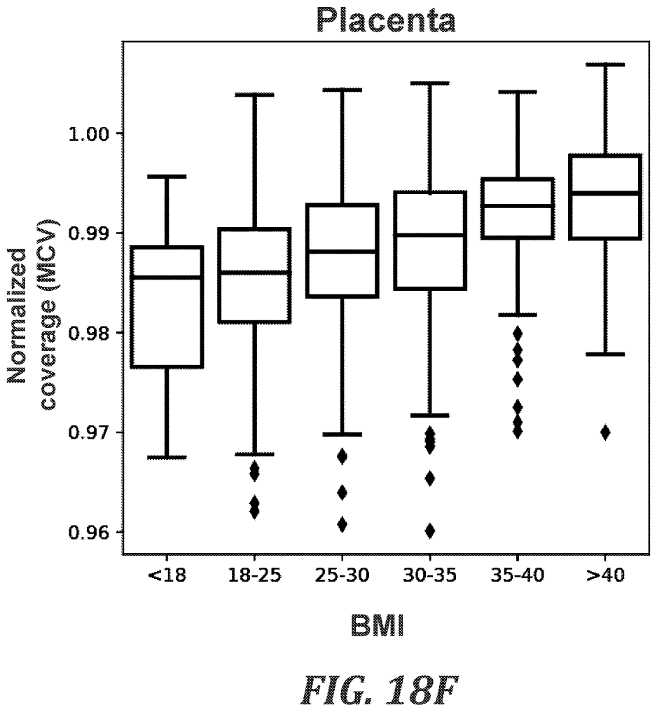

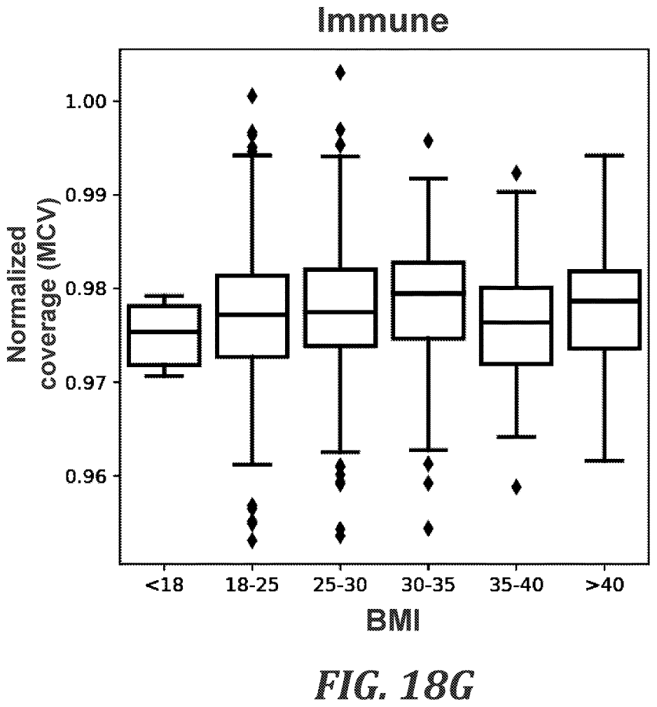

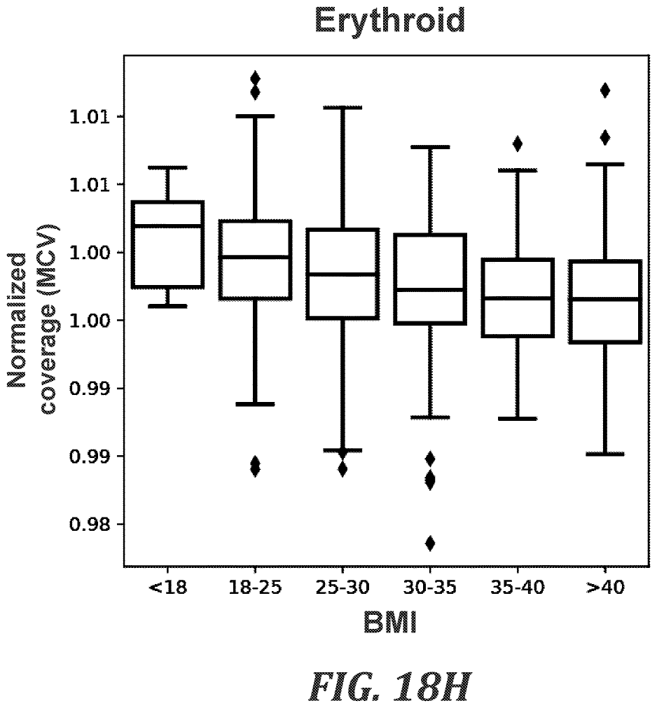

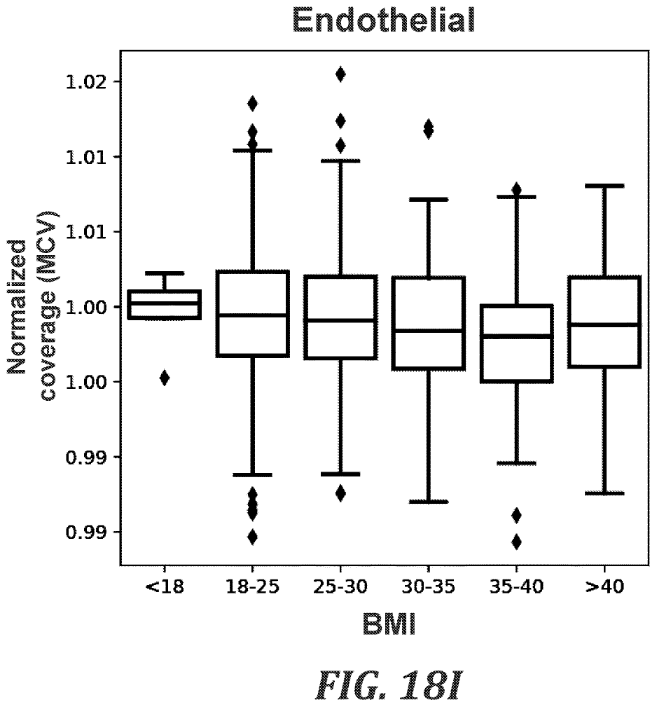

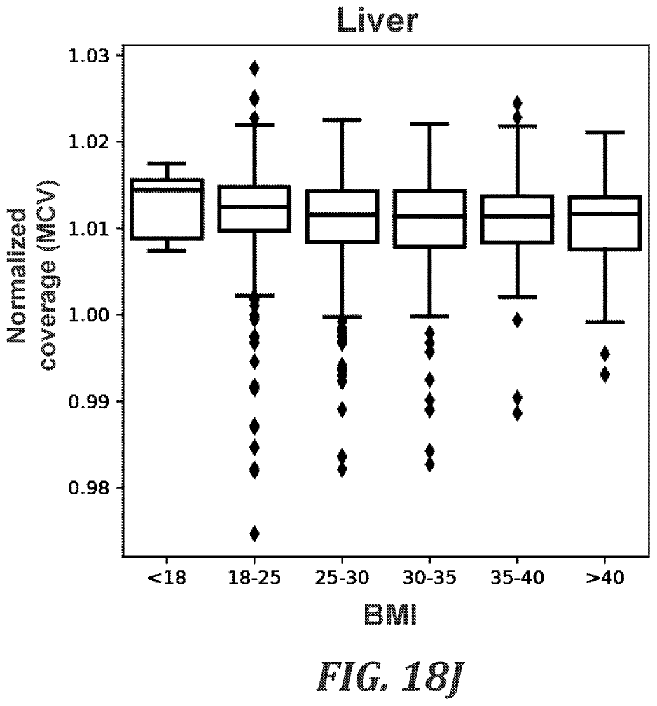

CfDNA nucleosome accessibility at open chromatin sites across tissue types was also evaluated to assess whether the signals are associated with maternal physiology (FIG. 16A-FIG. 16B). (First, differences in tissue-specific signals across the gestational period were measured. The immune signal was positively correlated with gestational age in weeks (GW) (n=985, immune NDR; Spearman's p=0.29, p=3.4e-19, FIG. 17A-FIG. 17J; FIG. 18A-FIG. 18J). However, placenta tissue signal was negatively correlated with GW, suggesting increased placental contribution with gestational age (n=985, Placenta MCV: Spearman's ρ=−0.19, p=2.5e-08, FIG. 17A-FIG. 17J; FIG. 18A-FIG. 18J). Decrease in endothelial tissue signal in the 3rd trimester was observed (n=985, Endothelial NDR: Mann Whitney U Test, p=1.4e-4, FIG. 17A-FIG. 17J; FIG. 18A-FIG. 18J). When observing sub-nucleosomal tissue specific profiles the placenta tissue NDR was positively correlated with GW (n=985, Placenta NDR: Spearman's p=0.23, p=3.3e-12). These results were further corroborated when using tissue-specific sites derived from DNase-Seq.

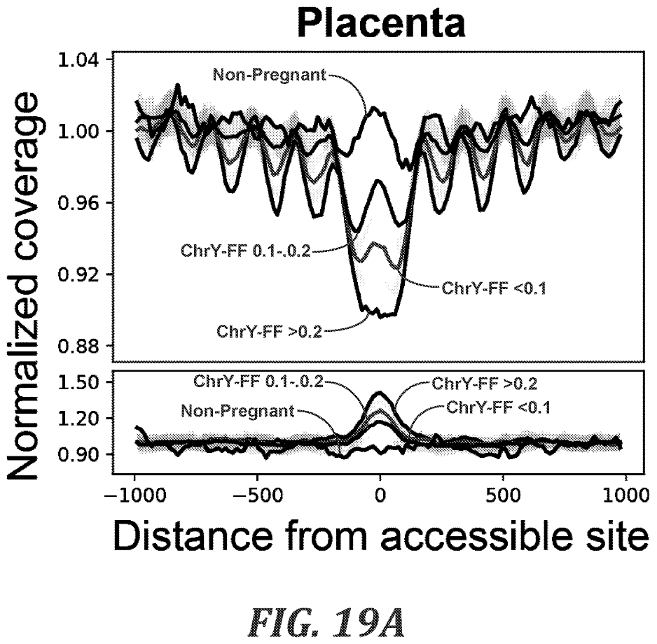

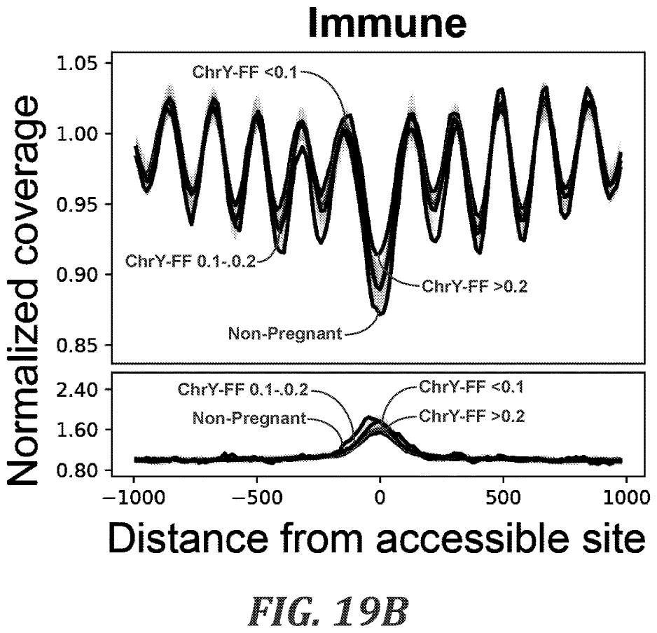

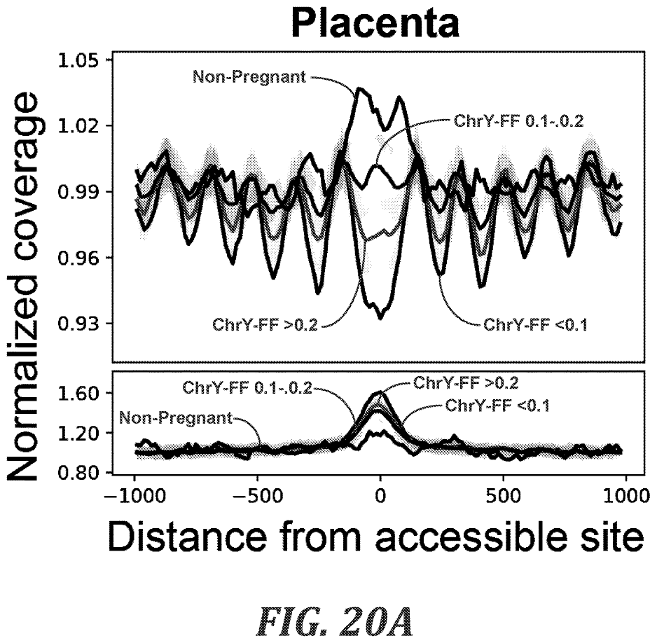

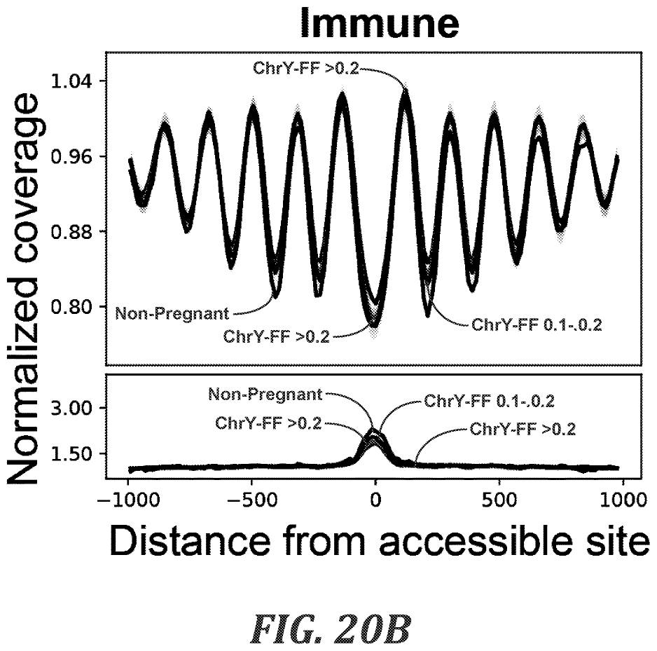

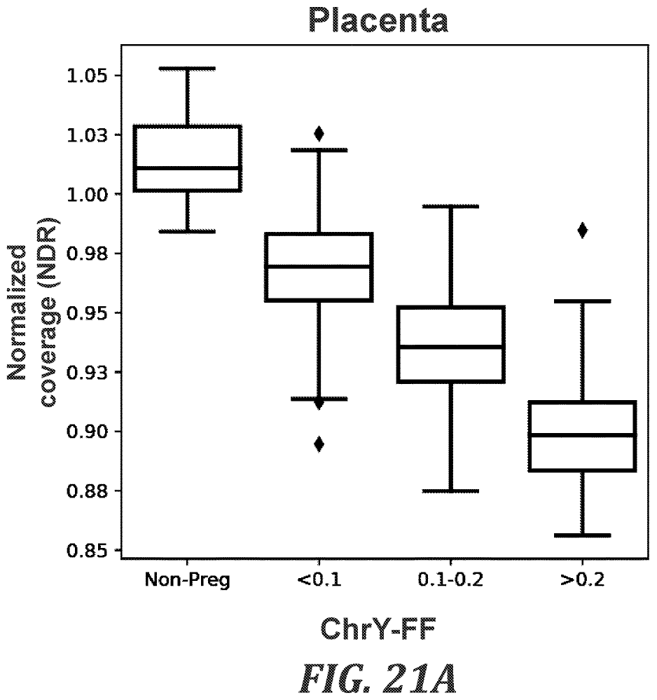

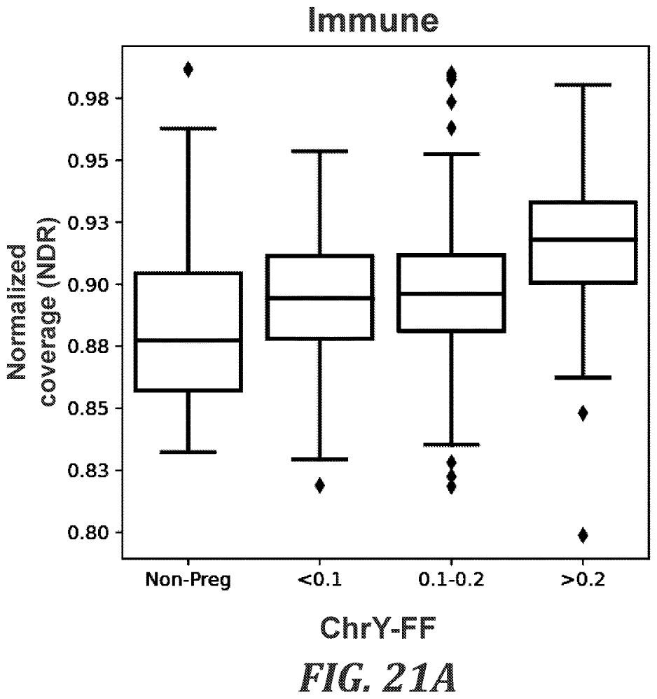

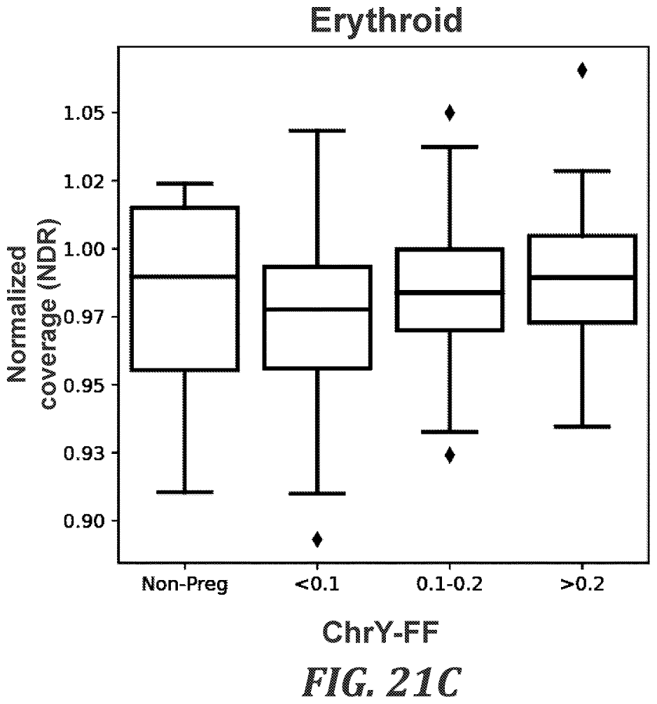

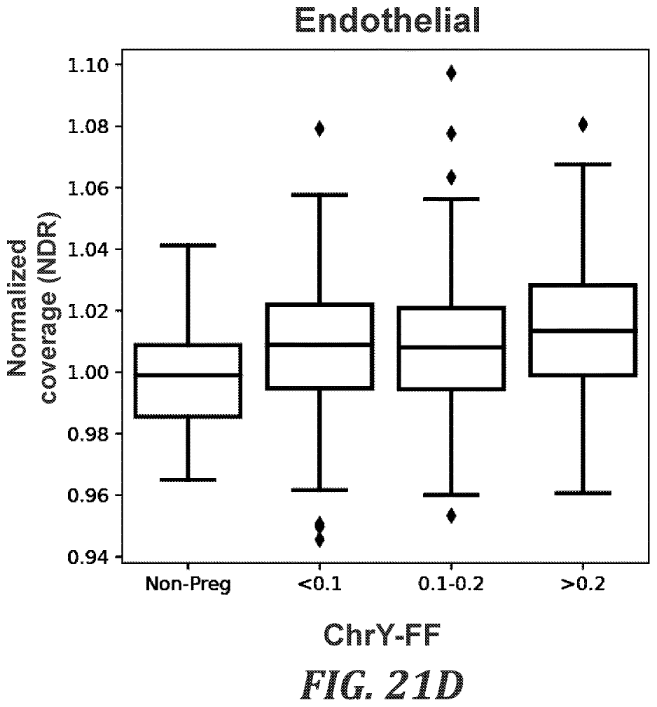

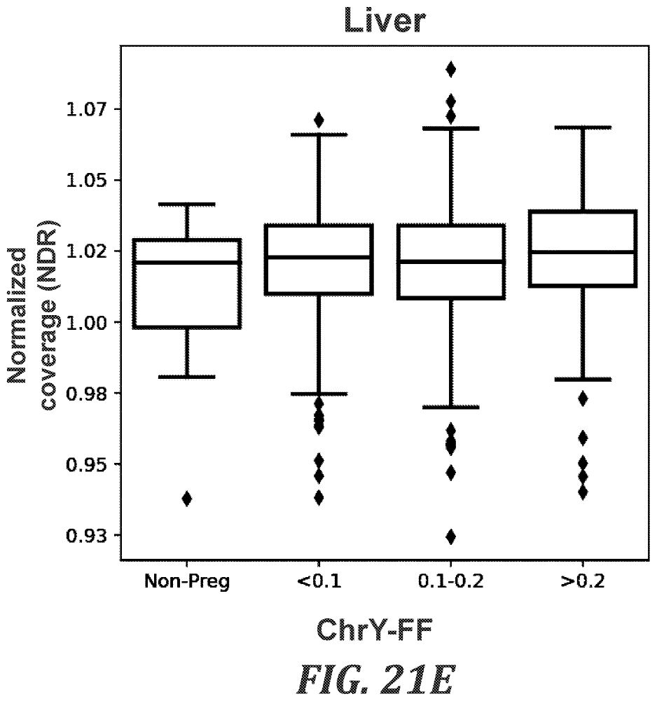

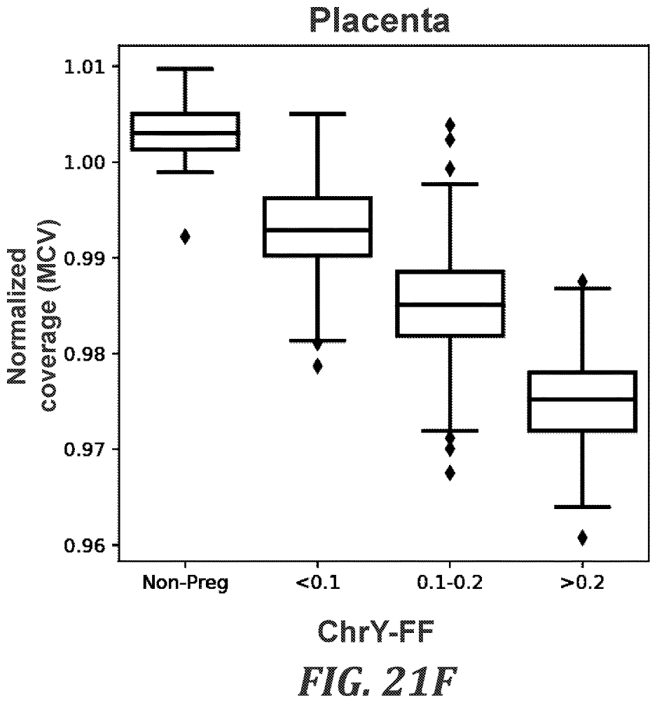

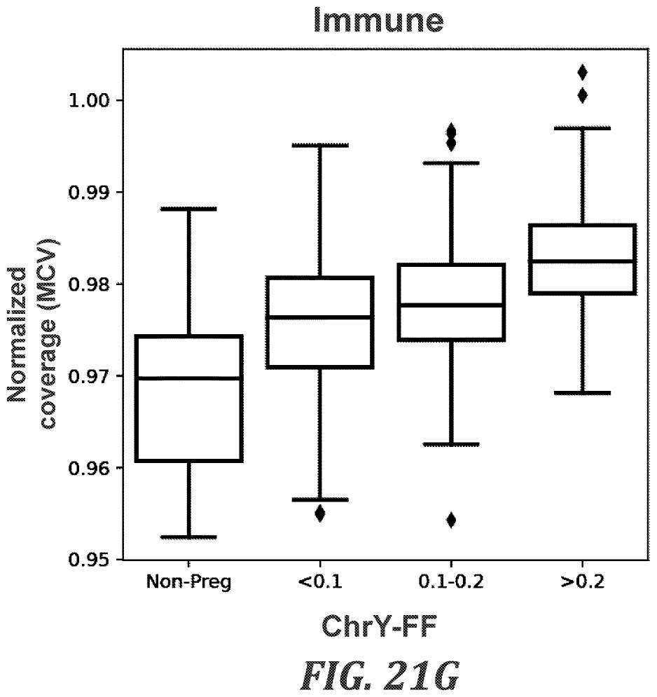

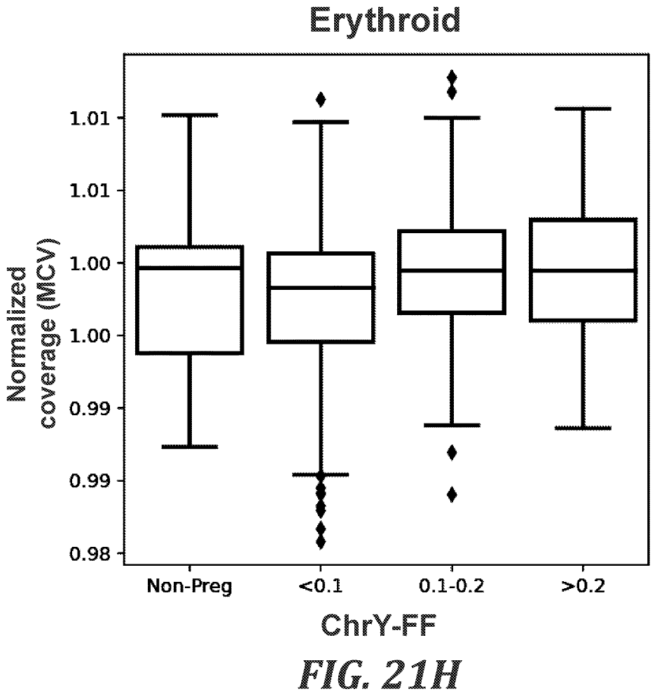

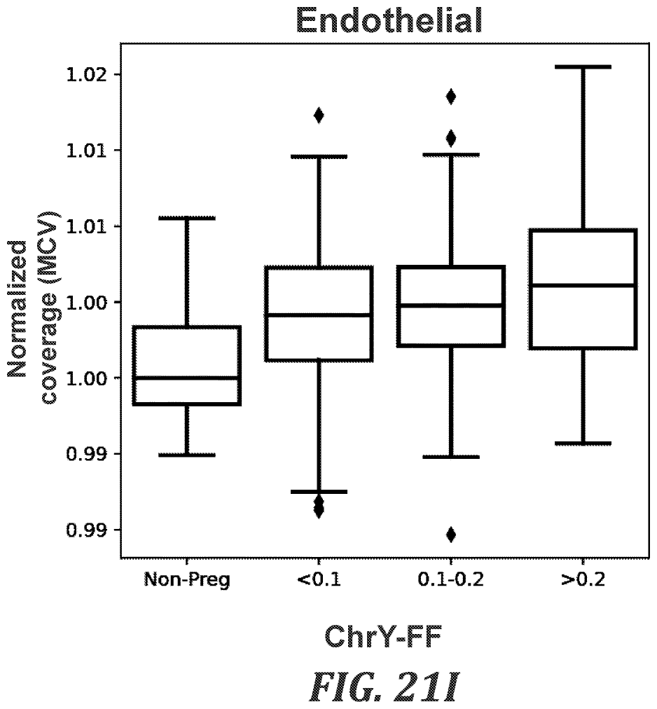

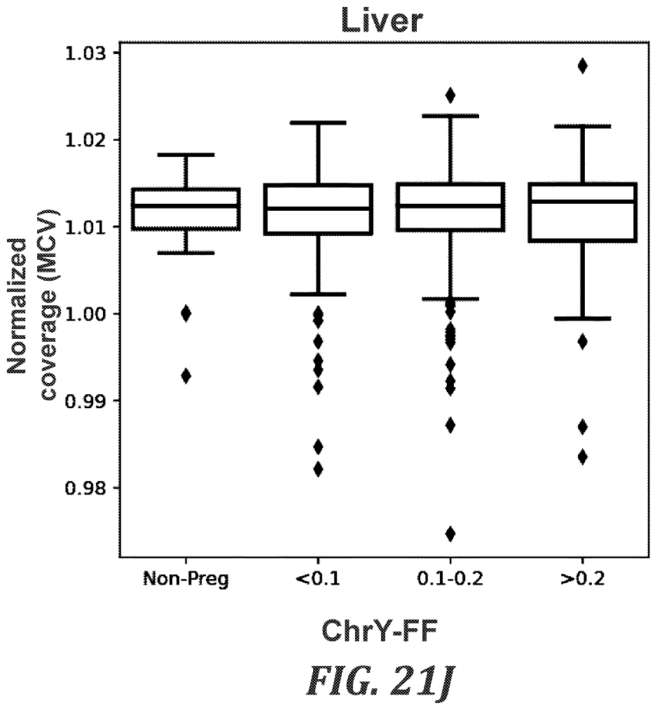

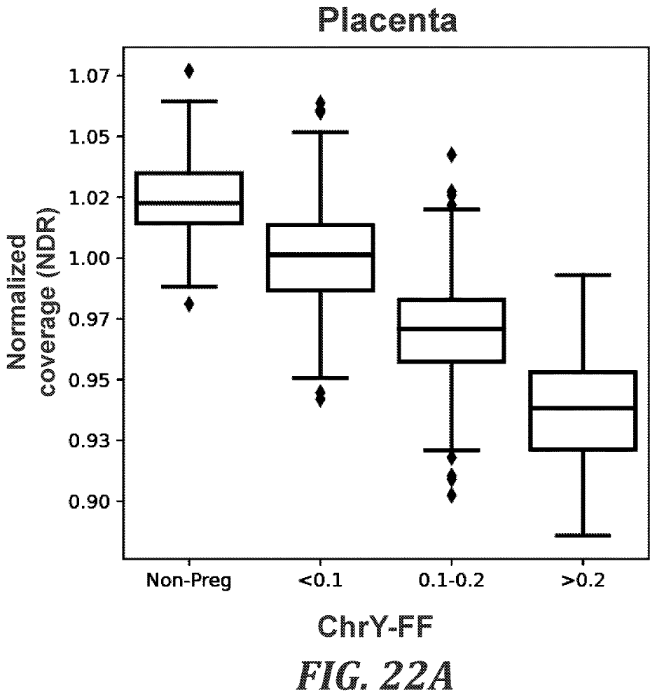

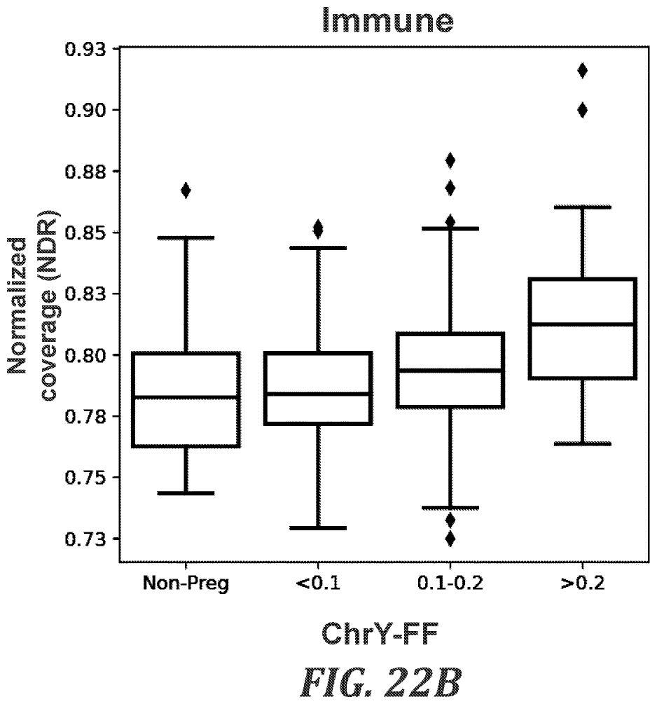

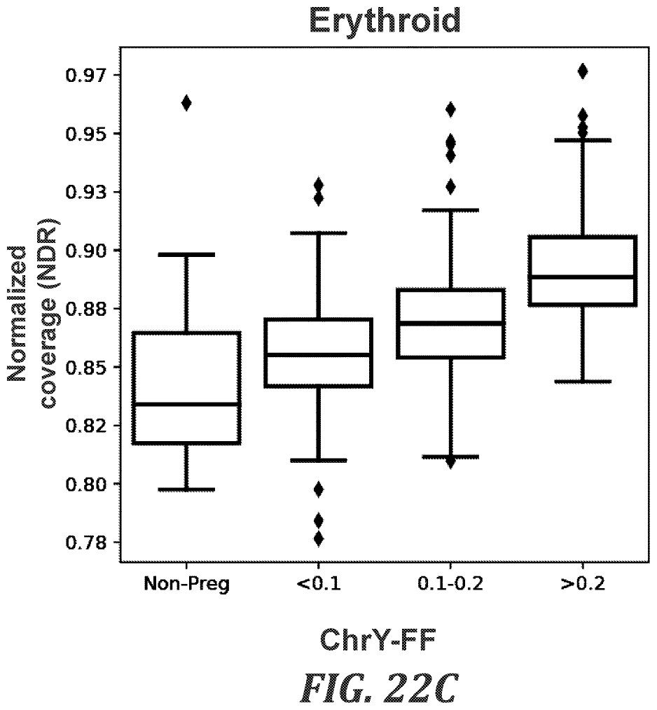

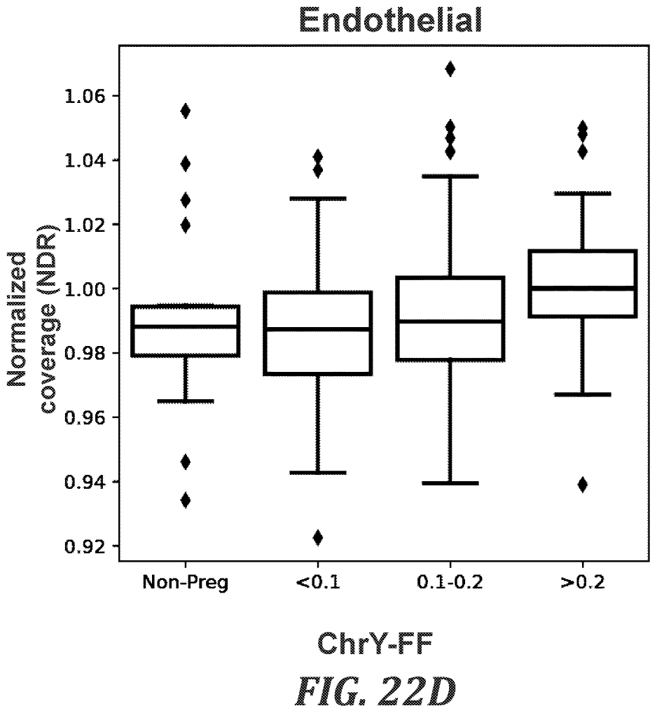

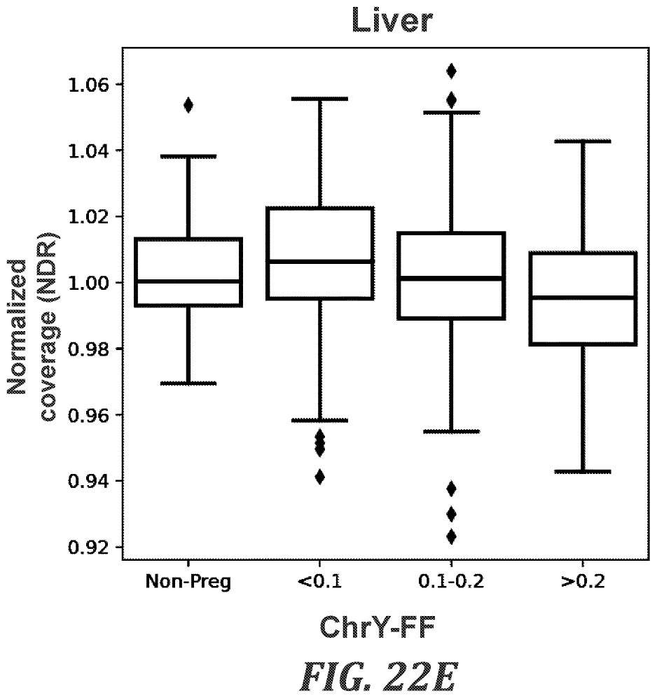

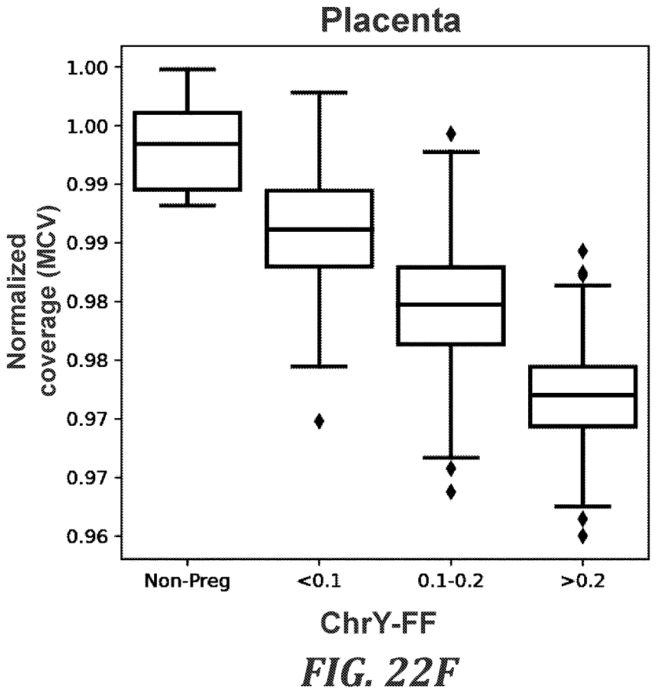

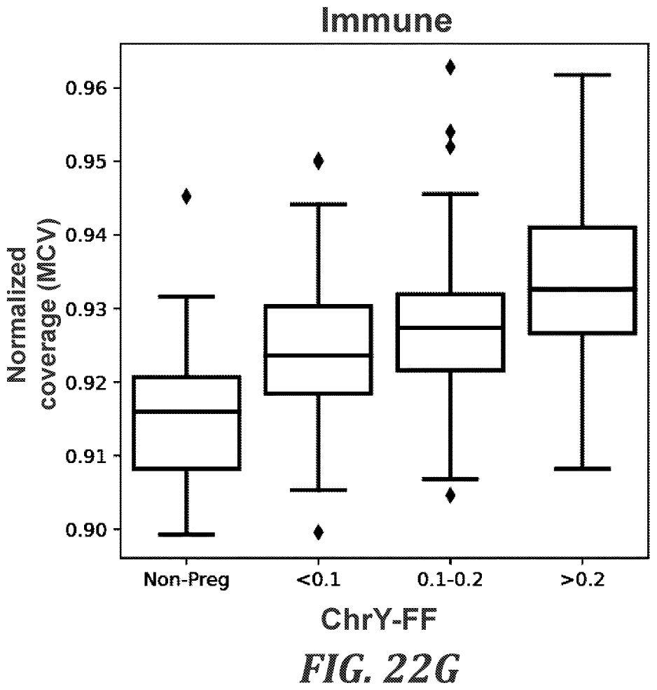

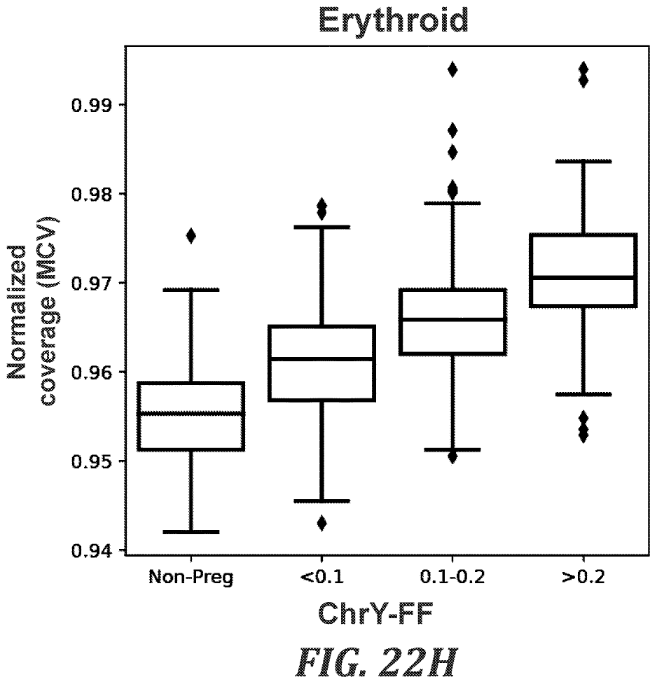

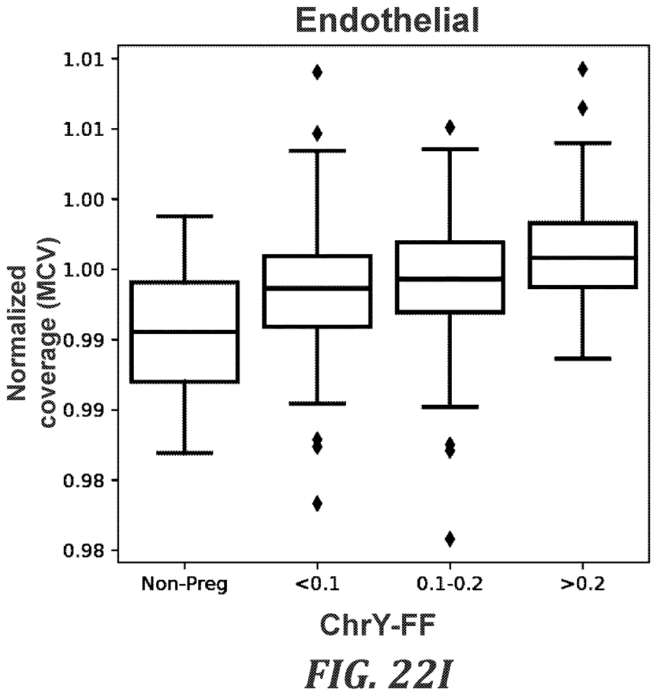

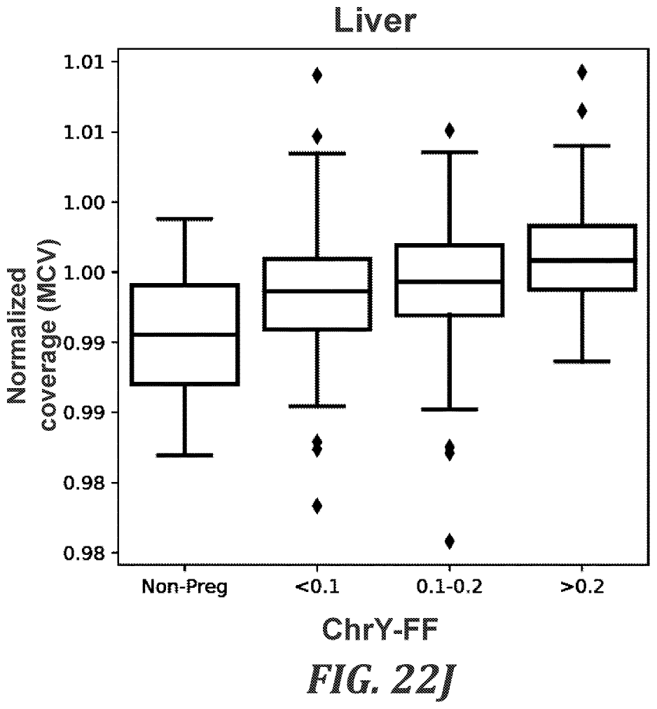

It was also investigated whether the nucleosome profiles of tissue-specific sites can inform the FF. It was observed that the placenta tissue-specific sites were correlated negatively (n=749, placenta NDR; Spearman's ρ=−0.79, p=1.5e-151, FIG. 19A, FIG. 19B, FIG. 21A-FIG. 21J) with FF determined by chrY-FF for samples with XY fetuses, as computed in clinical NIPS. However, immune and endothelial tissue-specific sites were positively correlated with ChrY-FF (n=749, immune NDR; Spearman's ρ=0.22, p=2.9e-8, endothelial MCV; Spearman's ρ=0.13, p=0.001, FIG. 20A, FIG. 20B, FIG. 21A-FIG. 21J) which could be due to the increasing FF. Additionally, the sub-nucleosome profiles for placenta were positively correlated with ChrY-FF (n=749, placenta NDR; Spearman's ρ=0.53, p-4.3e-53, FIG. 19A). This observation was also observed for tissue-specific sites derived from DNase-Seq (FIG. 22A-FIG. 22J).

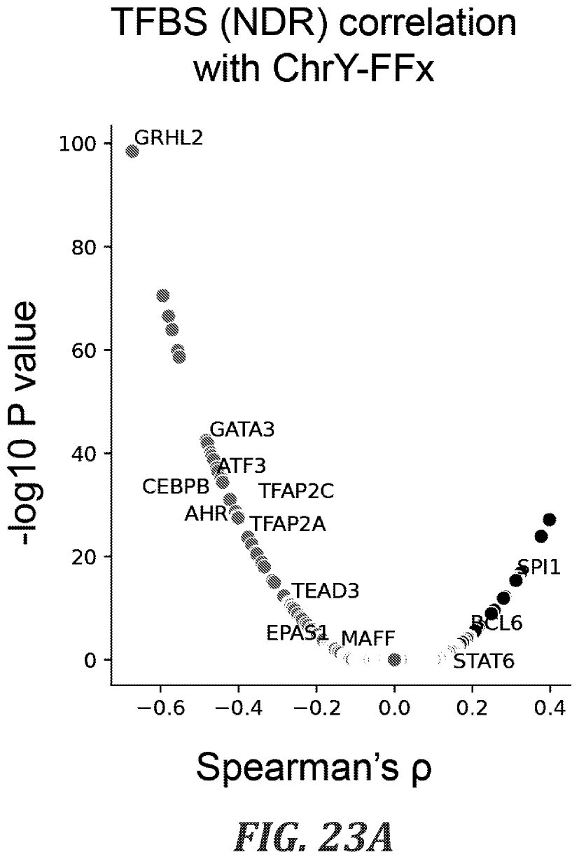

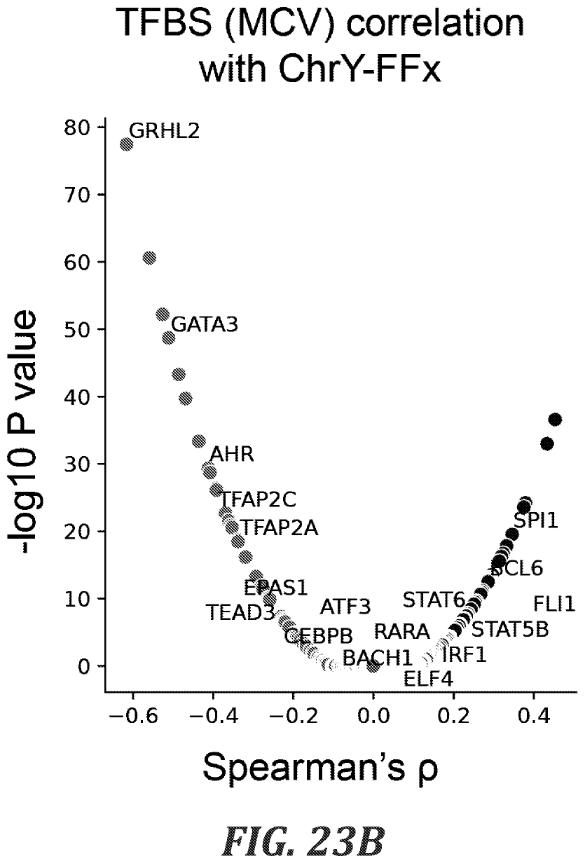

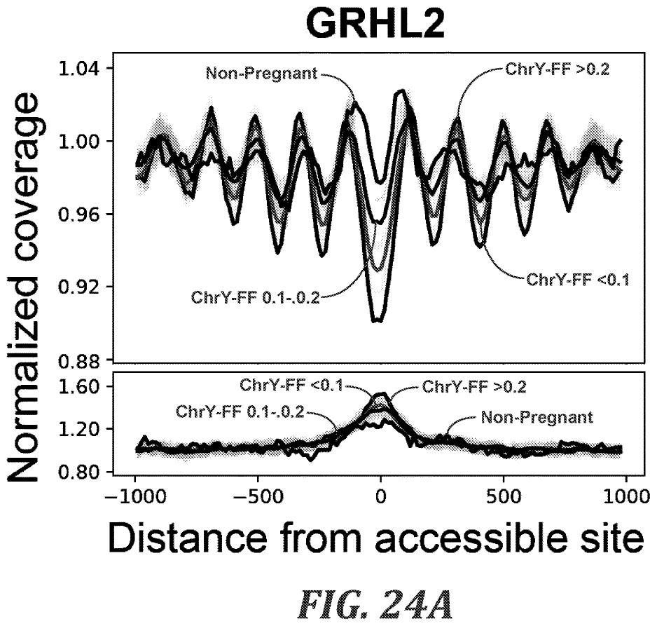

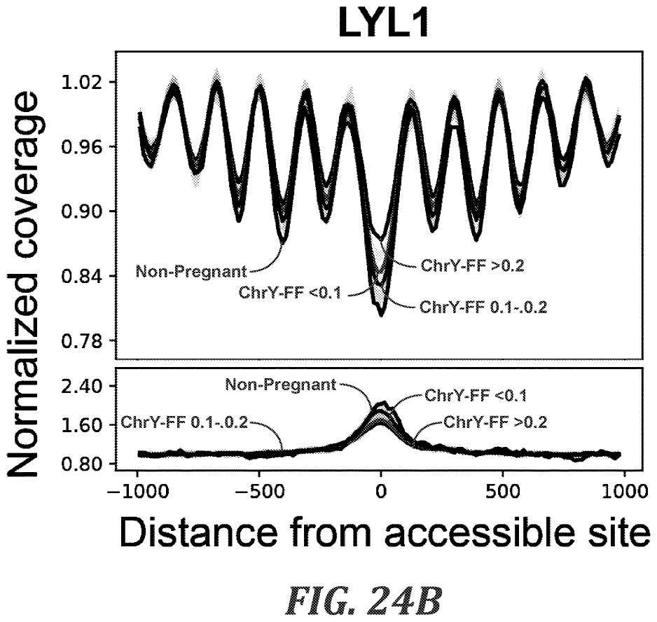

To infer transcriptional regulation, TFBSs were used. It was hypothesized that TFBS accessibility for some of the TFs would correlate with ChrY-FF. 82 TFs were identified with statistically significant negative correlation and 70 TFs with positive correlation with ChrY-FF. Gene set enrichment analysis (GSEA) revealed the negatively correlated TFs to be placental tissue related (for examples: TFAP2A, TFAP2C, CEBPB, EPAS1, MAFF, AHR, GATA3, GRHL2, ATF3, TEAD3). Grainy head-like 2 (GRHL2) TF, which had the highest correlation (n=749, NDR; Spearman's ρ=−0.68, p-3.0e-99, FIG. 23A-FIG. 23B; FIG. 24A-FIG. 24B), is known to be highly expressed in the trophoblast cells of placenta where it plays a crucial role in placental morphogenesis. Similarly, TEAD4, TEAD1, GATA3 & TFAP2A TFs have also been found to be expressed in placental tissue. Conversely, positively correlated TFs (for examples: LYL1, MECOM, RUNX1, NR4A1, STAT5B, SPI1, BCL6, ELF4, IRF1, RARA, STAT6, BACH1, FLI1) were found to be associated with blood cells (FIG. 23A-FIG. 23B; FIG. 24A-FIG. 24B). These results taken together highlight that tissue-specific accessible sites and TFBS coverage profiles inferred from sWGS of cfDNA can be used to identify tissues of origin.

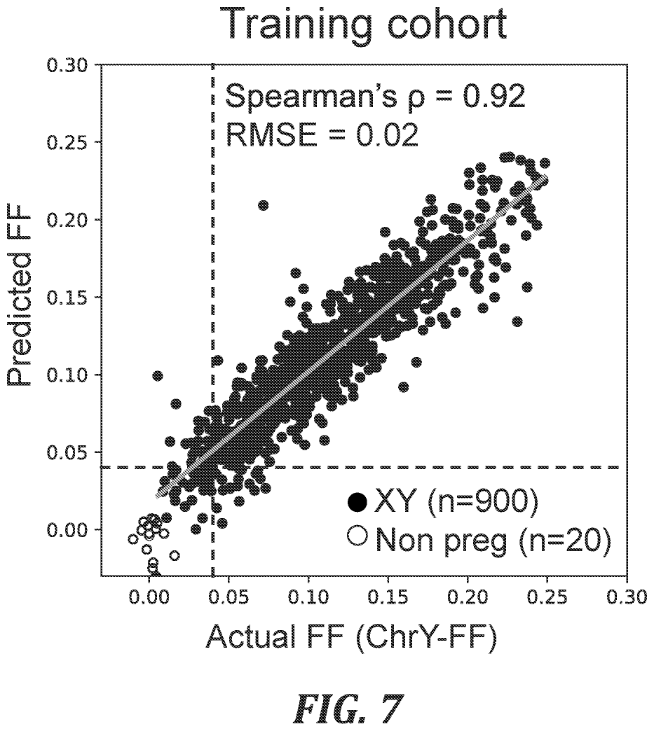

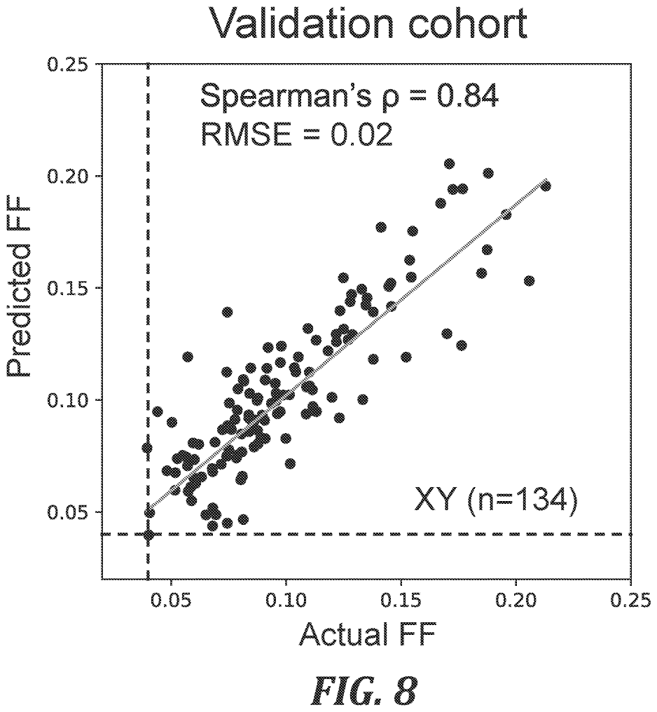

A regularized linear regression model was built to estimate the FF from NIPS data (henceforth called Griffin-FF). To further improve the detection of placental tissue bulk ATAC-seq (n=5) was performed to acquire placental tissue-specific accessible sites. To identify top features, values were compared for all tissue/cell-types and TFBS coverage profiles between non-pregnant controls (n=20) and pregnancy samples with high ChrY-FFx>0.25 (n=21) and identified 81 statistically significant features (Mann Whitney U Test, P-value <0.05). The most informative sites were placental tissue-specific sites determined from either bulk ATAC-seq, scATAC-seq or DNase-Seq (Mann Whitney U Test, P-value=0.00008). Supervised training was then performed using ChrY-FF as the ground truth for 900 male fetus NIPS samples in the training cohort. Using five-fold cross validation and bootstrapping (1000 iterations), a training performance was achieved of Spearman's ρ=0.92 (95% CI: 0.923-0.924) and root mean squared error (RMSE) of 0.021 (95% CI: 0.0207-0.0208) (FIG. 7). A final (locked) model was then trained on all 900 samples using the optimal hyperparameters. The final model was applied to estimate the Griffin-FF in an internal validation cohort (n=134) and achieved a performance of Spearman's ρ=0.84 and a RMSE of 0.02 (FIG. 8). Using the highest predicted Griffin-FF in non-pregnant cohort, the limit of detection was evaluated to be 0.008 (range:−0.037-0.008).

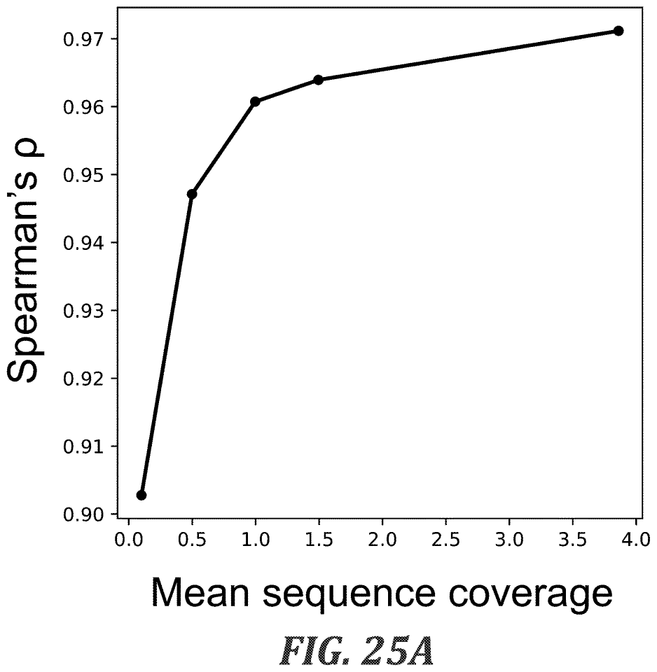

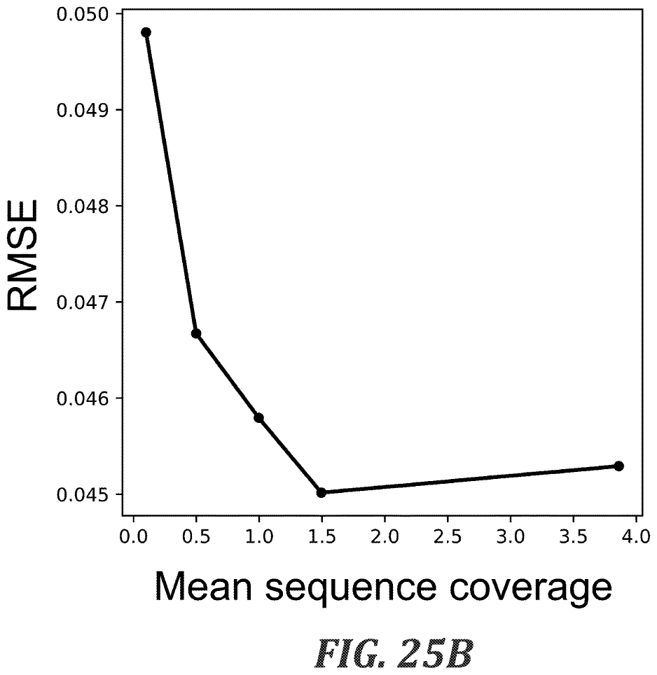

To further test the performance of the model, the Griffin-FF was estimated in 69 NIPS samples from an external cohort consisting of 21 male and 48 female fetuses. For this cohort, the FF had been estimated using SNPs from available parent-fetus trios, which provides an orthogonal ground truth for comparison (see Jiang, P. et al., “FetalQuantSD: accurate quantification of fetal DNA fraction by shallow-depth sequencing of maternal plasma DNA.” Npj Genomic Med. 1, 16013 (2016), hereby incorporated by reference herein in its entirety for all purposes.) Applying the locked model to estimate Griffin-FF, a performance was achieved with Spearman rho=0.97 and RMSE of 0.05 (FIG. 9). Because this external cohort of trios include fetuses from both sexes, this evaluation benchmarks the reliability of the model to estimate FF independently of fetal sex. Furthermore, since this dataset was sequenced to a median depth of 4×, in silico down-sampling was performed to 1.5×, 1×, 0.5× and 0.1× to test the effect of sequencing depth on Griffin-FF. The 69 samples were downsampled to 1.5×, 1×, 0.5× and 0.1× and a decrease in performance was observed with reducing sequence depth (FIG. 25A-FIG. 25B). These results suggest that nucleosome profiling from cfDNA can be used to accurately estimate FF from cfDNA.

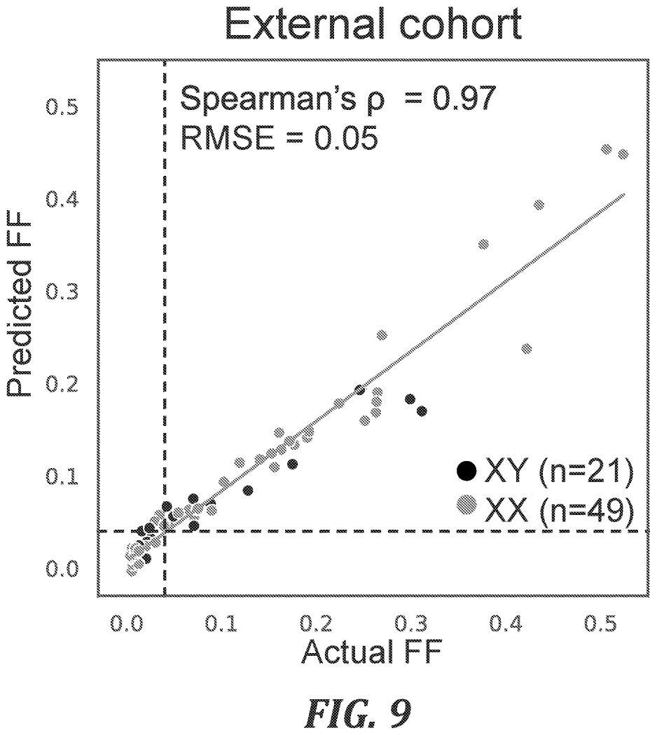

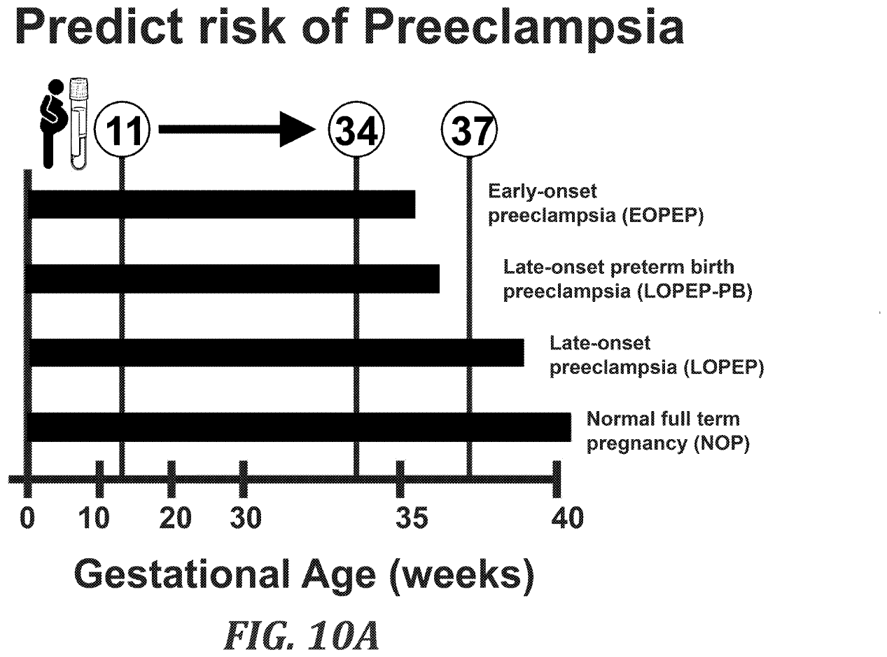

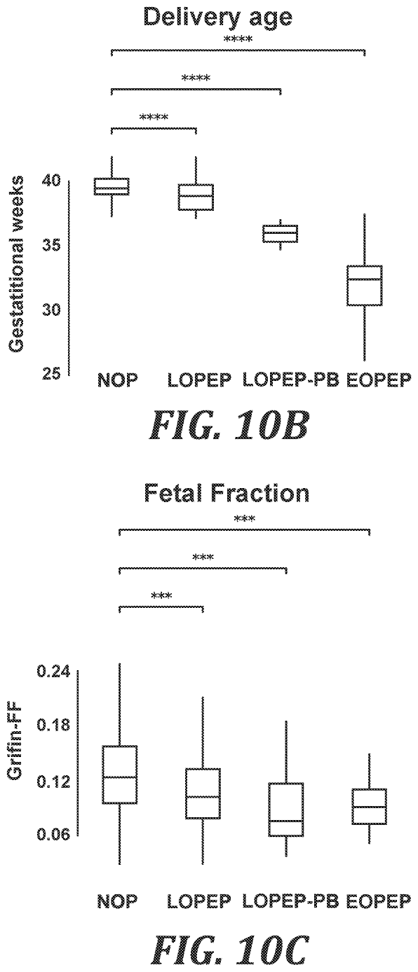

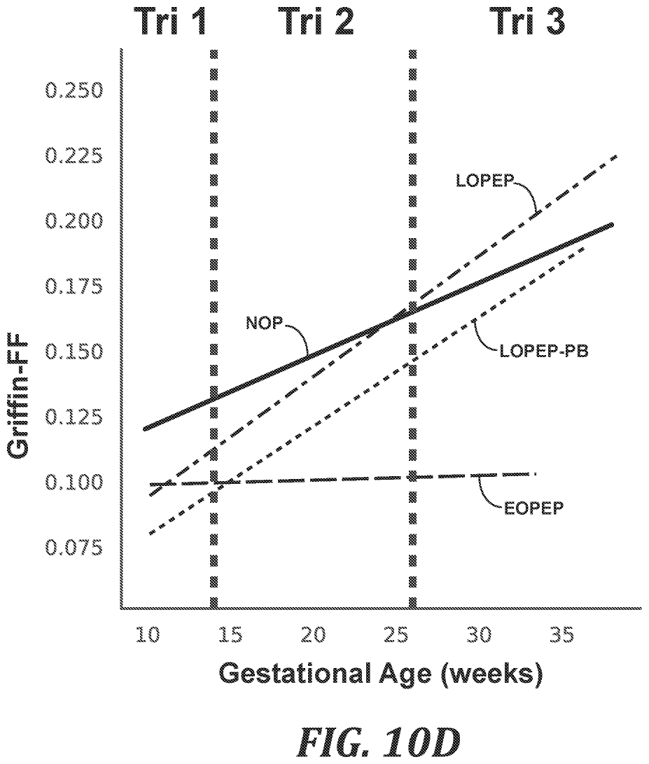

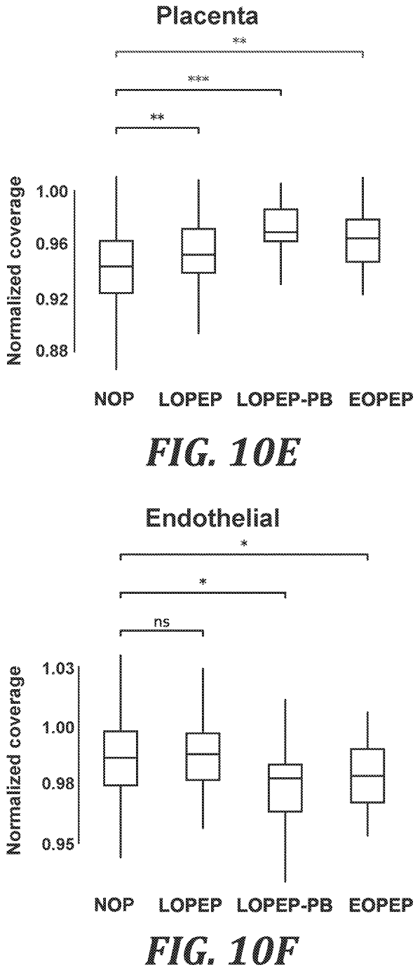

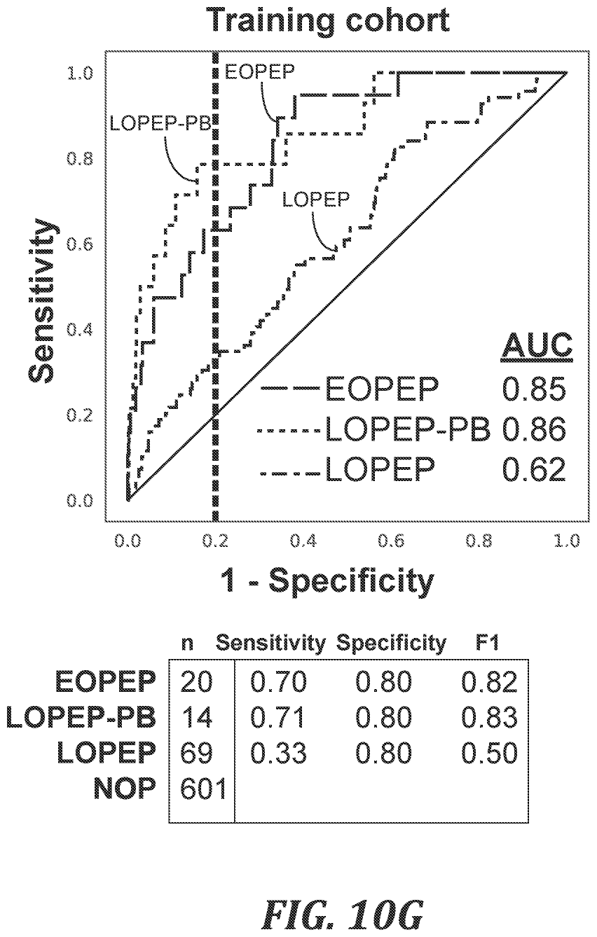

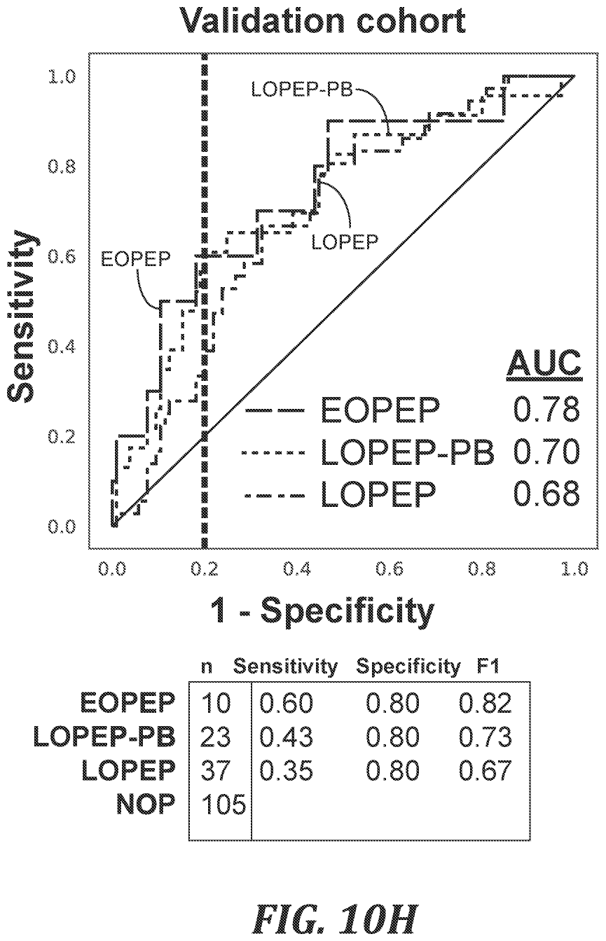

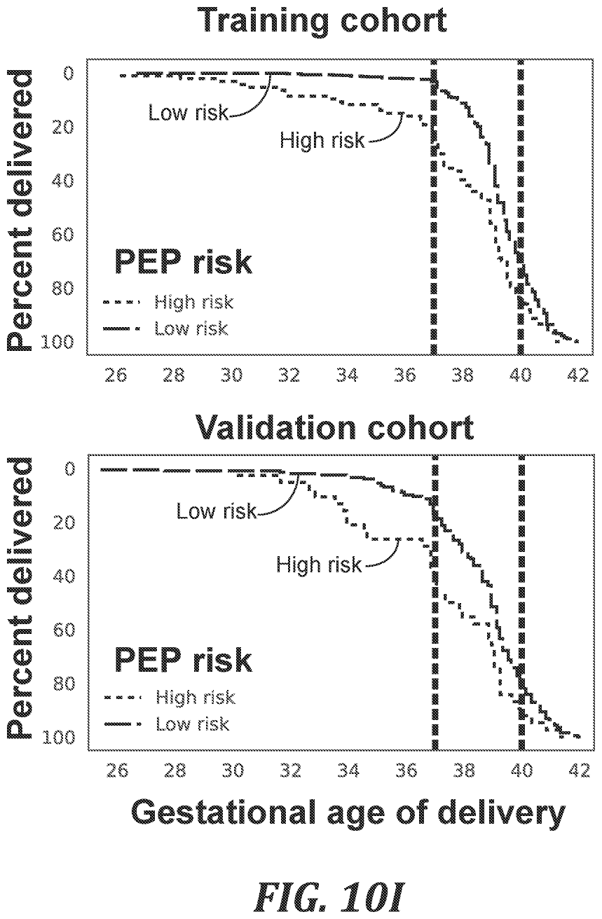

Nucleosome profiling of cfDNA from NIPS to predict preeclampsia was also tested. The utility of NIPS was investigated to determine whether it can be expanded to predict pregnancy complications including but not limited to PEP. 948 NIPS samples (median gestational age at draw 12.2 weeks, range 8-31 weeks) for which the pregnancy outcome was retrospectively determined were analyzed. 175 patients were identified who developed PEP and 773 patients who had a normal pregnancy (NOP). The PEP cohort was further classified into clinical subtypes based on gestational age at onset of hypertension (≤34 weeks or >34 weeks) and gestational age at delivery (<37 weeks ≥37 weeks) (FIG. 10A, FIG. 10B). These included early onset PEP (gestational age at onset≤34 weeks, EOPEP), late onset PEP (gestational age at onset >34 weeks, LOPEP) and LOPEP with preterm birth (gestational age at onset >34 weeks with delivery at <37 weeks [LOPEP-PB]) ([FIG. XXX, FIG. XXX]). Subset of NOP samples were further reviewed with additional exclusion criteria (absence of any maternal, fetal, or obstetric complications) to generate a set of reference controls (n=200). Furthermore, to perform early prediction of PEP using NIPS samples were considered (PEP n=117 & NOP n=666) collected early in pregnancy (≤16 weeks of gestation).

It was investigated whether FF as measured by nucleosome profiling (Griffin-FF) was different early in pregnancy (≤16 weeks of gestation). To this end, the FF estimator was retrained excluding samples that were part of this retrospective cohort. It was found that Griffin-FF was significantly reduced in all PEP subtypes when compared to reference NOP (Mann Whitney U Test, EOPEP; −value=0.0012, LOPEP-PB; P-value=5.7e-4, LOPEP; P-value=0.0013, FIG. 10C). Across gestational age, the Griffin-FF was observed to be significantly lower for EOPEP throughout all three trimesters (FIG. 10D). These results suggest that FF in PEP remain lower throughout the course of the pregnancy ([FIG. XXX]). These findings suggest that reduced FF early in pregnancy is associated with an increased risk for PEP.

BMI and blood pressure (systolic and diastolic) available at the time of NIPS testing were found to be significantly higher in PEP patients (Mann Whitney U Test, systolic; EOPEP; P-value=1.5e-6, LOPEP-PB; P-value=1.7e-4, LOPEP; P-value=2.7e-6, diastolic; EOPEP; P-value=1.1e-4, LOPEP-PB; P-value=3.4e-5, LOPEP; P-value=6.3e-5, BMI; EOPEP; P-value=1.2e-4, LOPEP-PB; P-value=0.041, LOPEP; P-value=0.001) compared to reference normal pregnancy.

Placenta tissue specific nucleosome chromatin profiles were significantly higher (Mann Whitney U Test, Placental NDR; EOPEP; P-value=0.0071, LOPEP-PB; P-value=5.9e-4, LOPEP; P-value=0.027, FIG. 10E) early in pregnancy (≤16 weeks gestational age), further supporting reduced FF observed in PEP. Previous work has shown that preeclampsia is characterized by endothelial dysfunction. It was observed that for EOPEP and LOPEP-PB, samples had lower coverage profiles (Mann Whitney U Test, Endothelial MCV; EOPEP; P-value=0.017, LOPEP-PB; P-value=0.1, FIG. 10F), suggesting endothelial damage ([Figure XXXX]). To ensure these findings were not affected by FF, the Griffin-FF was used as a covariate, and this result was still significant (ANCOVA. EOPEP; P-value=0.047, LOPEP-PB; P-value=0.27). No other tissue types were observed to be significantly different between PEP subtypes and normal pregnancy. These results were further validated when using tissue specific sites derived from DNase-Seq. Taken together these results suggest that reduced placental cfDNA fraction and increased endothelial cfDNA fraction early in pregnancy (≤16 weeks gestation) are associated with increased risk for PEP.

A machine learning model was trained for the early prediction of PEP (≤16 weeks gestation, n=777, NOP=592 and PEP=103). The model incorporated three feature sets: 1) placental, endothelial tissue specific nucleosome features (NDR & MCV) and Griffin-FF, 2) systolic blood pressure (SBP) and diastolic blood pressure (DBP), and 3) combination of set 1 and 2. An ensemble model was trained to distinguish NOP and PEP, and the model's performance was evaluated using a cross-validation approach. The performance of the model was further evaluated for the different subtypes. The model predicted EOPEP with an AUC 0.85 (area under curve) (95% confidence interval (CI), 0.83-0.86) and sensitivity of 0.70 at specificity 0.80. For LOPEP-PB the model had similar performance with an AUC 0.86 (CI, 0.85-0.87) and sensitivity of 0.71 at 0.80 specificity (FIG. 10G). The model for LOPEP had an AUC 0.62 (CI, 0.61-0.62) and sensitivity of 0.33 at 0.80 specificity. The final model trained using all samples in training cohort was then applied to a validation cohort (≤16 weeks of gestation, n=175, NOP=105 and PEP=70). The validation performance achieved AUC values of 0.78, 0.70, and 0.68 for EOPEP, LOPEP, and LOPEP-PB, respectively (FIG. 10H). The model's risk score (i.e. probability of PEP) also showed a statistically significant inverse correlation with the gestational age of delivery (Spearman's ρ=−0.18, P-value=1.8e-6 for training cohort, Spearman's ρ=−0.24, p=2.2e-4 for validation cohort, FIG. 10I). Additionally, samples classified as high risk by the model had a range of 20-30% chance of having a preterm delivery ([Figure XXX]). These results highlight that using cfDNA nucleosome early in pregnancy (≤16 weeks of gestation) can predict risk for preeclampsia.

### DISCUSSION

Placental-derived cfDNA can be readily accessed from maternal circulation to detect placental specific signatures in a potentially diagnostic manner, making it analogous to a liquid biopsy of the placenta. This biology is currently leveraged for prenatal aneuploidy screening and is ideal for refinements to glean additional information about pregnancy health. Techniques have been presented for sex-independent FF determination and early pregnancy PEP prediction from an early pregnancy blood sample using a computational innovation that reveals an epigenetic layer (nucleosome positioning) within the WGS data generated for NIPS.

FF assessment is used for aneuploidy determination; however, indirect assessments of this placental-derived fraction lack accuracy-particularly assays relying on size-discrepancy differences in pregnancies with female fetuses. The Griffin computational tool was applied, and a new fetal sex-independent tool for FF determination was developed. This methodology performs direct assessment of the placental contribution and demonstrates excellent correlation with both internal and external validation samples, indicating reliability regardless of fetal sex, and quantification of the placental-derived fraction of cfDNA (i.e., the FF).

This feature, the Griffin-predicted FF, serves as a feature in the PEP prediction model. By discerning placenta-specific nucleosome coverage profiles early in pregnancy (≤16 weeks gestation), and incorporating basic clinical factors (blood pressure, BMI), a predictive model was developed that can predict severe phenotypes of PEP (early-onset and those complicated by preterm birth) with promising accuracy.

The innovations reported herein highlight that nucleosome profiling from cfDNA can be utilized to expand the prenatal screening assay to include PEP screening early in pregnancy.

In the preceding description, numerous specific details are set forth to provide a thorough understanding of various embodiments of the present disclosure. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The order in which some or all of the blocks appear in each method flowchart should not be deemed limiting. Rather, one of ordinary skill in the art having the benefit of the present disclosure will understand that actions associated with some of the blocks may be executed in a variety of orders not illustrated, or even in parallel.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

The above description of illustrated embodiments of the disclosure, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

### EXAMPLES

While general features of the disclosure are described and shown and particular features of the disclosure are set forth tin the claims, the following non-limiting examples relate to features, and combinations of features, that are explicitly envisioned as being part of the disclosure. The following non-limiting examples contain elements that are modular and can be combined with each other in any number, order, or combination to form a new non-limiting example, which can itself be further combined with other non-limiting examples.
Example 1. A computer-implemented method of enhancing sequence read data from a cell-free DNA (cfDNA) sample from a subject for predicting a pregnancy-related condition, the method comprising: receiving, by a computing system, sequence read data, wherein the sequence read data includes a plurality of fragment reads; determining, by the computing system, a coverage profile based on the sequence read data for a plurality of informative sites associated with specific tissue types, cell types, or cell states; determining, by the computing system, a predicted fetal fraction based on the sequence read data; determining, by the computing system, a set of features based on the predicted fetal fraction and a set of features based on the coverage profile; and generate, by the computing system, a prediction of a presence of or an absence of the pregnancy-related condition by providing at least features from the set of features based on the predicted fetal fraction and the set of features based on the coverage profile as input to at least one machine learning model trained to predict a probability of future onset of the pregnancy-related condition based on the features.
Example 2. The computer-implemented method of example 1, wherein determining the predicted fetal fraction based on the sequence read data includes performing a method as recited in any one of example 42 to example 59.
Example 3. The computer-implemented method of any one of example 1-2, further comprising filtering the fragment reads to include nucleosome-sized profile reads, subnucleosomal-sized profile reads, and dinucleosomal-sized profile reads.
Example 4. The computer-implemented method of example 3, wherein the nucleosome-sized profile reads have a size in a range from 100 bp to 200 bp, wherein the subnucleosomal-sized profile reads have a size in a range less than 100 bp, and wherein the dinucleosomal-sized profile reads have a size in a range greater than 200 bp.
Example 5. The computer-implemented method of example 4, wherein the nucleosome-sized profile reads have a size in a range from 120 bp to 180 bp.
Example 6. The computer-implemented method of any one of example 4-5, wherein the subnucleosomal-sized profile reads have a size in a range from 35 bp to 80 bp.
Example 7. The computer-implemented method of any one of example 4-6, wherein the dinucleosomal-sized profile reads have a size in a range from 300 bp to 400 bp.
Example 8. The computer-implemented method of any one of example 1-7, wherein the coverage profile is generated for windows flanking each informative site.
Example 9. The computer-implemented method of example 8, wherein the windows flanking each informative site have a size in a range from ±500 bp from the informative site to ±5,000 bp from the informative site.
Example 10. The computer-implemented method of example 9, wherein the windows flanking each informative site have a size of ±1000 bp from the informative site.
Example 11. The computer-implemented method of any one of example 1-10, wherein the fragment reads are generated from target-enriched sequencing.
Example 12. The computer-implemented method of example 11, wherein target-enriched sequencing includes hybridization capture sequencing or amplification-based sequencing.
Example 13. The computer-implemented method of any one of example 1-12, wherein the fragment reads are generated from low-coverage sequencing.
Example 14. The computer-implemented method of example 13, wherein genomic coverage provided by the low-coverage sequencing is greater or equal to 0.1× fold coverage of a whole genome.
Example 15. The computer-implemented method of any one of example 1-14, wherein each fragment read has a fragment length and a GC content indicating a percentage of bases in the fragment read that are G or C, and wherein determining the coverage profile for the plurality of informative sites associated with the pregnancy-related condition includes: determining GC bias values for each fragment read based on the fragment length and the GC content of the fragment read; and generating an adjusted coverage profile that is adjusted for GC bias using the sequence read data and the GC bias values.
Example 16. The computer-implemented method of example 15, wherein determining the set of features based on the coverage profile includes determining at least one of a nucleosome-depleted region (NDR) score and a mean coverage value (MCV) score for at least one informative site.
Example 17. The computer-implemented method of example 16, wherein determining the NDR score for the at least one informative site includes: determining, based on the coverage profile, a mean coverage value over a first range of distance from the informative site.
Example 18. The computer-implemented method of example 17, wherein the first range of distance from the informative site has a size in a range of ±1 bp to ±250 bp from the informative site.
Example 19. The computer-implemented method of example 18, wherein the first range of distance from the informative site has a size of ±30 bp from the informative site.
Example 20. The computer-implemented method of any one of example 16-19, wherein determining the MCV score for the at least one informative site includes: determining, based on the coverage profile, a mean coverage value within a second range of distance from the informative site.
Example 21. The computer-implemented method of example 20, wherein the second range of distance from the informative site has a size in a range of ±250 bp to ±5000 bp from the informative site.
Example 22. The computer-implemented method of example 21, wherein the second range of distance from the informative site has a size of ±1000 bp from the informative site.
Example 23. The computer-implemented method of any one of example 16-22, wherein providing at least features from the set of features based on the predicted fetal fraction and the set of features based on the coverage profile as input to at least one machine learning model includes providing the NDR scores and the MCV scores as features from the set of features based on the coverage profile, and providing the predicted fetal fraction as the feature from the set of features based on the predicted fetal fraction.
Example 24. The computer-implemented method of any one of example 1-23, further comprising determining a set of clinical features based on clinical values for the subject; wherein providing at least features from the set of features based on the predicted fetal fraction and the set of features based on the coverage profile as input to at least one machine learning model includes providing the set of clinical features as input to the at least one machine learning model.
Example 25. The computer-implemented method of example 24, wherein the clinical values include a set of blood pressure values.
Example 26. The computer-implemented method of example 25, wherein the set of blood pressure values include at least one of a systolic blood pressure value and a diastolic blood pressure value.
Example 27. The computer-implemented method of any one of example 24-26, wherein the clinical values include a body mass index (BMI) value.
Example 28. The computer-implemented method of any one of example 24-27, wherein the at least one machine learning model includes: at least one machine learning model configured to accept the set of features based on the predicted fetal fraction and the set of features based on the coverage profile as input; at least one machine learning model configured to accept the set of clinical features as input; and at least one machine learning model configured to accept the features based on the predicted fetal fraction, the set of features based on the coverage profile, and the set of clinical features as input.
Example 29. The computer-implemented method of any one of example 1-28, wherein the at least one machine learning model includes at least one of an L1-normalized logistic regression model, an L2-normalized logistic regression model, a random forest model, or an XGBoost model.
Example 30. The computer-implemented method of any one of example 1-29, wherein the at least one machine learning model includes at least one of a probabilistic model, a latent state probabilistic model, a Bayesian statistical model, or a generative probabilistic model.
Example 31. The computer-implemented method of any one of example 1-30, wherein the informative sites include at least one of sites associated with placental tissues and sites associated with endothelial tissues.
Example 32. The computer-implemented method of any one of example 1-31, wherein the informative sites include at least one of a transcription factor binding site (TFBS) or an open chromatin site.
Example 33. The computer-implemented method of any one of example 1-32, wherein the informative sites are tissue-specific sites unique to at least one human tissue type.
Example 34. The computer-implemented method of example 33, wherein the tissue-specific sites inform prediction of tissues-of-origin.
Example 35. The computer-implemented method of any one of example 1-34, wherein the cfDNA sample was collected from a subject at or before 16 weeks gestation.
Example 36. The computer-implemented method of any one of example 1-34, wherein the cfDNA sample was collected from a subject after 16 weeks gestation.
Example 37. The computer-implemented method of any one of example 1-36, wherein the pregnancy-related condition is preeclampsia.
Example 38. The computer-implemented method of example 37, wherein predicting the probability of future onset of the pregnancy-related condition based on the features includes predicting a severity of preeclampsia.
Example 39. The computer-implemented method of example 38, wherein predicting the severity of preeclampsia includes predicting a normal pregnancy, early-onset preeclampsia, late-onset preeclampsia, or late-onset preeclampsia with preterm birth.
Example 40. The computer-implemented method of any one of example 1-39, wherein the pregnancy-related condition is small neonatal size for gestational age.
Example 41. The computer-implemented method of any one of example 1-40, wherein the pregnancy-related condition is intrauterine growth restriction.
Example 42. A computer-implemented method of enhancing sequence read data from a cell-free DNA (cfDNA) sample from a subject for predicting a fetal fraction of cfDNA contained in the sample, the method comprising: receiving, by a computing system, sequence read data, wherein the sequence read data includes a plurality of fragment reads; determining, by the computing system, a coverage profile for a plurality of informative sites associated with fetal fraction determination; determining, by the computing system, a set of features based on the coverage profile; and generate, by the computing system, a prediction of a fetal fraction by providing features from the set of features as input to at least one machine learning model trained to predict the fetal fraction based on the features.
Example 43. The computer-implemented method of example 42, wherein the plurality of informative sites associated with fetal fraction determination includes one or more tissue-specific informative sites.
Example 44. The computer-implemented method of example 43, wherein the one or more tissue-specific informative sites include at least one of a placental tissue-specific informative site or an immune cell tissue informative site.
Example 45. The computer-implemented method of any one of example 42-44, wherein the plurality of informative sites associated with fetal fraction determination includes one or more transcription factor binding sites.
Example 46. The computer-implemented method of example 45, wherein the one or more transcription factor binding sites include at least one transcription factor binding site associated with a GRHL2 transcription factor, a TEAD4 transcription factor, a TEAD1 transcription factor, a GATA3 transcription factor, a TFAP2A transcription factor, a LYL1 transcription factor, a MECOM transcription factor, a RUNX1 transcription factor, or an NR4A1 transcription factor.
Example 47. The computer-implemented method of any one of example 42-46, wherein the coverage profile is generated for windows flanking each informative site.
Example 48. The computer-implemented method of example 47, wherein the windows flanking each informative site have a size in a range from ±500 bp from the informative site to ±5,000 bp from the informative site.
Example 49. The computer-implemented method of example 48, wherein the windows flanking each informative site have a size of ±1000 bp from the informative site
Example 50. The computer-implemented method of any one of example 42-49, wherein each fragment read has a fragment length and a GC content indicating a percentage of bases in the fragment read that are G or C, and wherein determining the coverage profile for the plurality of informative sites includes: determining GC bias values for each fragment read based on the fragment length and the GC content of the fragment read; and generating an adjusted coverage profile that is adjusted for GC bias using the sequence read data and the GC bias values.
Example 51. The computer-implemented method of any one of example 42-50, wherein determining the set of features based on the coverage profile includes determining a nucleosome-depleted region (NDR) score and a mean coverage value (MCV) score for each informative site.
Example 52. The computer-implemented method of example 51, wherein determining the NDR score for each informative site includes, for each informative site: determining, based on the coverage profile, a mean coverage value over a first range of distance from the informative site.
Example 53. The computer-implemented method of example 52, wherein the first range of distance from the informative site has a size in a range from ±1 bp to ±250 bp from the informative site.
Example 54. The computer-implemented method of example 53, wherein the first range of distance from the informative site has a size of ±30 bp from the informative site
Example 55. The computer-implemented method of any one of example 51-54, wherein determining the MCV score for each informative site includes, for each informative site: determining, based on the coverage profile, a mean coverage value within a second range of distance from the informative site.
Example 56. The computer-implemented method of example 55, wherein the second range of distance from the informative site has a size in a range from ±250 bp to ±5000 bp from the informative site.
Example 57. The computer-implemented method of example 56, wherein the second range of distance from the informative site has a size of ±1000 bp from the informative site.
Example 58. The computer-implemented method of any one of example 51-57, wherein providing features from the set of features as input to the at least one machine learning model includes providing the NDR scores and MCV scores as features from the set of features.
Example 59. The computer-implemented method of any one of example 42-58, wherein the at least one machine learning model includes a regularized linear regression model.
Example 60. The computer-implemented method of any one of example 42-59, wherein the cfDNA sample was collected from a subject at or before 16 weeks gestation.
Example 61. The computer-implemented method of any one of example 42-59, wherein the cfDNA sample was collected from a subject after 16 weeks gestation.
Example 62. A computing system configured to perform a method as recited in any one of example 1-61.
Example 63. A non-transitory computer-readable medium having computer-executable instructions stored thereon that, in response to execution by a computing system, cause the computing system to perform actions of a method as recited in any one of example 1-61.

1. A computer-implemented method of enhancing sequence read data from a cell-free DNA (cfDNA) sample from a subject for predicting a pregnancy-related condition, the method comprising:
receiving, by a computing system, sequence read data, wherein the sequence read data includes a plurality of fragment reads;
determining, by the computing system, a coverage profile based on the sequence read data for a plurality of informative sites associated with specific tissue types, cell types, or cell states;
determining, by the computing system, a predicted fetal fraction based on the sequence read data;
determining, by the computing system, a set of features based on the predicted fetal fraction and a set of features based on the coverage profile; and
generating, by the computing system, a prediction of a presence of or an absence of the pregnancy-related condition by providing at least features from the set of features based on the predicted fetal fraction and the set of features based on the coverage profile as input to at least one machine learning model trained to predict a probability of future onset of the pregnancy-related condition based on the features.

2. (canceled)

3. The computer-implemented method of claim 1, further comprising filtering the fragment reads to include nucleosome-sized profile reads, subnucleosomal-sized profile reads, and dinucleosomal-sized profile reads.

4-7. (canceled)

8. The computer-implemented method of claim 1, wherein the coverage profile is generated for windows flanking each informative site,
optionally wherein the windows flanking each informative site have a size in a range from ±500 bp from the informative site to ±5,000 bp from the informative site.

9-12. (canceled)

13. The computer-implemented method of claim 1, wherein the fragment reads are generated from low-coverage sequencing,
optionally wherein genomic coverage provided by the low-coverage sequencing is greater or equal to 0.1× fold coverage of a whole genome.

14. (canceled)

15. The computer-implemented method of claim 1, wherein each fragment read has a fragment length and a GC content indicating a percentage of bases in the fragment read that are G or C, and wherein determining the coverage profile for the plurality of informative sites associated with the pregnancy-related condition includes:
determining GC bias values for each fragment read based on the fragment length and the GC content of the fragment read; and
generating an adjusted coverage profile that is adjusted for GC bias using the sequence read data and the GC bias values.

16. The computer-implemented method of claim 15, wherein determining the set of features based on the coverage profile includes determining at least one of a nucleosome-depleted region (NDR) score and a mean coverage value (MCV) score for at least one informative site,
optionally wherein determining the NDR score for the at least one informative site includes determining, based on the coverage profile, a mean coverage value over a first range of distance from the informative site,
optionally wherein determining the MCV score for the at least one informative site includes determining, based on the coverage profile, a mean coverage value within a second range of distance from the informative site.

17-22. (canceled)

23. The computer-implemented method of claim 16, wherein providing at least features from the set of features based on the predicted fetal fraction and the set of features based on the coverage profile as input to at least one machine learning model includes providing the NDR scores and the MCV scores as features from the set of features based on the coverage profile, and providing the predicted fetal fraction as the feature from the set of features based on the predicted fetal fraction.

24. The computer-implemented method of claim 1, further comprising determining a set of clinical features based on clinical values for the subject;
wherein providing at least features from the set of features based on the predicted fetal fraction and the set of features based on the coverage profile as input to at least one machine learning model includes providing the set of clinical features as input to the at least one machine learning model.

25. The computer-implemented method of claim 24, wherein the clinical values include a set of blood pressure values and/or a body mass index (BMI) value.

26. (canceled)

27. (canceled)

28. The computer-implemented method of claim 24, wherein the at least one machine learning model includes:
at least one machine learning model configured to accept the set of features based on the predicted fetal fraction and the set of features based on the coverage profile as input;
at least one machine learning model configured to accept the set of clinical features as input; and
at least one machine learning model configured to accept the features based on the predicted fetal fraction, the set of features based on the coverage profile, and the set of clinical features as input.

29. (canceled)

30. (canceled)

31. The computer-implemented method of claim 1, wherein the informative sites include at least one of sites associated with placental tissues and sites associated with endothelial tissues.

32. The computer-implemented method of claim 1, wherein the informative sites include at least one of a transcription factor binding site (TFBS) or an open chromatin site.

33-36. (canceled)

37. The computer-implemented method of claim 1, wherein the pregnancy-related condition is preeclampsia,
optionally wherein predicting the probability of future onset of the pregnancy-related condition based on the features includes predicting a severity of preeclampsia,
optionally wherein predicting the severity of preeclampsia includes predicting a normal pregnancy, early-onset preeclampsia, late-onset preeclampsia, or late-onset preeclampsia with preterm birth.

38-41. (canceled)

42. A computer-implemented method of enhancing sequence read data from a cell-free DNA (cfDNA) sample from a subject for predicting a fetal fraction of cfDNA contained in the sample, the method comprising:
receiving, by a computing system, sequence read data, wherein the sequence read data includes a plurality of fragment reads;
determining, by the computing system, a coverage profile based on the sequence read data for a plurality of informative sites associated with fetal fraction determination;
determining, by the computing system, a set of features based on the coverage profile; and
generating, by the computing system, a prediction of a fetal fraction by providing features from the set of features as input to at least one machine learning model trained to predict the fetal fraction based on the features.

43. The computer-implemented method of claim 42, wherein the plurality of informative sites associated with fetal fraction determination includes one or more tissue-specific informative sites,
optionally wherein the one or more tissue-specific informative sites include at least one of a placental tissue-specific informative site or an immune cell tissue informative site.

44. (canceled)

45. The computer-implemented method of claim 42, wherein the plurality of informative sites associated with fetal fraction determination includes one or more transcription factor binding sites.

46-50. (canceled)

51. The computer-implemented method of claim 42, wherein determining the set of features based on the coverage profile includes determining a nucleosome-depleted region (NDR) score and a mean coverage value (MCV) score for each informative site,
optionally wherein determining the NDR score for each informative site includes, for each informative site, determining, based on the coverage profile, a mean coverage value over a first range of distance from the informative site,
optionally wherein determining the MCV score for each informative site includes, for each informative site, determining, based on the coverage profile, a mean coverage value within a second range of distance from the informative site.

52-57. (canceled)

58. The computer-implemented method of claim 51, wherein providing features from the set of features as input to the at least one machine learning model includes providing the NDR scores and MCV scores as features from the set of features.

59-61. (canceled)

62. A computing system configured to perform a method as recited in claim 1.

63. A non-transitory computer-readable medium having computer-executable instructions stored thereon that, in response to execution by a computing system, cause the computing system to perform actions of a method as recited in claim 1.
